(19) United States
(12) Patent Application Publication
(10) Pub. No.: US 2025/0325601 A1
(43) Pub. Date: Oct. 23, 2025

(54) COMPOSITION OF ORAL ADMINISTRATION OBTAINED FROM LYSATES OF PROBIOTIC MICROORGANISMS FOR USE AS AN ANTI-AGING AGENT

(71) Applicant: SIGNIFER SL, Madrid (ES)

(72) Inventors: Maria Jesús GUARDIA ALBA, Granada (ES); Juan MOSCOSO DEL PRADO UCELAY, Tres Cantos (ES)

(21) Appl. No.: 19/121,012

(22) PCT Filed: Oct. 13, 2023
(86) PCT No.: PCT/EP2023/078516

§ 371 Date: Apr. 14, 2025

(30) Foreign Application Priority Data
Oct. 14, 2022 (ES) P202230884

(51) Int. Cl. A61K 35/742; A61K 31/375; A61K 31/525; A61K 31/593; A61K 33/04; A61K 33/30; A61K 33/32; A61K 35/00; A61K 35/744; A61K 35/745; A61K 35/747; A61K 36/064; A61P 39/06

(52) U.S. Cl. CPC A61K 35/742; A61K 31/375; A61K 31/525; A61K 31/593; A61K 33/04; A61K 33/30; A61K 33/32; A61K 35/744; A61K 35/745; A61K 35/747; A61K 36/064; A61P 39/06; A61K 2035/115

(57) ABSTRACT

A composition of oral administration for use in the prevention and/or treatment of oxidative stress related with aging, comprising lysates of probiotic microorganisms in the form of dry powder of the genus Bacillus; Bifidobacterium; Lactobacillus; Saccharomyces and Streptococcus.

### TECHNICAL FIELD OF THE INVENTION

The present invention describes a composition comprising lysates of probiotic microorganisms in the form of dry powder as an anti-aging agent and is highly safe even with a long period of consumption. Such a composition is used as a food supplement for the prevention and delay of aging in humans.

Thus, the present invention is encompassed in the technical field of therapies whose object it is to delay or prevent the development of decreased energy, change of appearance in humans and animals due to aging.

### BACKGROUND

Average life expectancy has increased rapidly in recent decades according to the World Health Organization (2018), which reflects the good development that society has been experiencing. Although this increase is undoubtedly positive, a new challenge arises because aging is directly related to cognitive, biological and physical degeneration, which increases the risk of suffering from certain diseases. Age-related diseases have become the greatest threats to health in the twenty-first century.

Aging is a universal multifactorial and progressive intrinsic process characterized by being degenerative and accompanied by a progressive loss of function. According to the World Health Organization, the functional capacity of a person begins to decline after he has reached adulthood, then producing aging that can be defined as a biological process by which living beings undergo a series of structural and functional changes that appear over time and are not the result of diseases or accidents. When you get older, joint problems can appear, affecting flexibility, loss of muscle mass causing fatigue, and reduction of bone mineral density, increasing the likelihood of fractures. The rate of decline in a person's functional capacity appears to be determined, at least in part, by their behavior and the environmental living conditions to which he or she is exposed throughout their life. Influential factors are diet, physical activity, and exposure to risks such as smoking, the harmful consumption of alcohol, or exposure to toxic substances. One of the most frequent recommendations to counteract, mitigate and/or slow down the effects of aging as much as possible and improve healthy life expectancy is that people perform adequate physical activity, and follow a healthy diet based on vegetables, fruits, whole grains, foods with a lot of fiber, and sources of protein without fat, such as fish, with natural antioxidants and low saturated fat and salt.

Another theory explaining the aging process is the accumulation of reactive oxygen species that damage proteins, lipids and DNA by accumulating in cells. When the efficiency of endogenous antioxidant systems decreases, the presence of antioxidants is not enough to compensate reactive oxygen species, which generates oxidative stress that plays an important role in aging and age-related diseases such as certain types of cancer, diabetes and heart failure, and in the pathogenesis of gastrointestinal diseases, among others. Numerous studies have shown that resistance to oxidative stress is crucial for staying healthy and reducing the adverse effects of aging. Consequently, nutritional interventions using food-grade antioxidant compounds or food products are considered as an option to help improve health and quality of life in the elderly. However, there is currently no type of therapy that has been shown to be really effective in delaying the aging process.

Aging in people occurs due to a multitude of exogenous and endogenous factors that also largely seem to interact with each other, so that the problem arises of finding a simple administration product that mitigates or delays the aging process by extending the healthy life expectancy in people.

There are publications that have developed different aspects on the dynamism of the intestinal microbiota and its variations over the years, as well as other factors that can alter it and contribute to human aging.

That is why, faced with the technical problem posed by current treatments, the sector is actively working to develop treatments and compositions that can be administered orally to delay the effects of aging. As proof of this, the following patent documents are cited.

U.S. Pat. No. 8,492,353B2 provides an anti-aging composition containing at least one selected member of the group consisting of ascorbic acid, a derivative of ascorbic acid and a salt thereof (A), and a substance related to purine nucleic acid (B) and which is capable of effectively retarding skin aging, in particular relieving skin pigmentation. The present invention also provides a method to enhance the anti-aging action of ascorbic acids. The invention provides an anti-aging composition.

Patent application US2020345694A1 concerns an anti-aging composition containing cytochalasin D or SAG, and a method of selecting an anti-aging substance, and provides a new anti-aging substance and a new method of discovering a new anti-aging substance.

Patent application JP2003155234A describes an anti-aging composition comprising a methyl group donor compound, particularly betaine and, optionally, further comprising one or more types of substances selected from a group consisting of choline, methionine and carnitine. The composition suppresses the increase of homocysteine in the blood and prevents the aging of the human body caused by the increase in homocysteine, both in oral and percutaneous administration.

Nevertheless, none of the documents that are in the prior art disclose a stable composition that has a function of preventing and delaying aging by oral ingestion, as described in the present document, that is composed of dry bacterial lysates.

It is also worth mentioning the Spanish patent applications P201930242 and P201930280, patent applications of the inventors, whose content is incorporated here for reference, which describe a modulating composition of the human intestinal microbiome obtained from lysates of probiotic microorganisms and their method of obtainment, as well as a food supplement comprising the composition, which is useful in the prevention and treatment of disorders caused, or at least encouraged, by intestinal dysbiosis of the microbiota in humans.

Therefore, the present invention aims to solve the problems of the prior art by using a composition of oral administration obtained from lysates of probiotic microorganisms, for use in the prevention and delay of aging in humans.

### BRIEF DESCRIPTION OF THE INVENTION

In a first aspect, the present invention relates to the composition of oral administration to prevent and delay aging, that comprises lysates of probiotic microorganisms in the form of dry powder, in the following amount in percentage by weight with respect to the total:
5% to 30% of bacterial lysates of the genus Bacillus;
3% to 25% of bacterial lysates of the genus Bifidobacterium;
15% to 35% of bacterial lysates of the genus Lactobacillus;
30% to 65% of bacterial lysates of the genus Saccharomyces;
1.5% to 10% of bacterial lysates of the genus Streptococcus;
for use in the prevention and/or treatment of oxidative stress related with aging.

In a further aspect, the invention relates to the use of the composition object of the invention as a food supplement for the prevention and delay of aging in animal or human cells.

In a further aspect, the invention relates to a pharmaceutical composition comprising an effective pharmaceutical amount of the composition, in accordance with the first aspect of the invention, and a pharmaceutically acceptable excipient.

In a further aspect, the invention relates to the use of a composition of oral administration to prevent and delay aging, that comprises lysates of probiotic microorganisms in the form of dry powder, in the following amount in percentage by weight with respect to the total:
5% to 30% of bacterial lysates of the genus Bacillus;
3% to 25% of bacterial lysates of the genus Bifidobacterium;
15% to 35% of bacterial lysates of the genus Lactobacillus;
30% to 65% of bacterial lysates of the genus Saccharomyces;
1.5% to 10% of bacterial lysates of the genus Streptococcus;

### BRIEF DESCRIPTION OF THE FIGURES

To complement the description and help render a better understanding of the invention, we have accompanied as an integral part of said description, a set of figures where, for illustrative and non-limiting purposes, graphs of the experimental results obtained in examples 1 to 4 that are collected in this document are presented.

FIG. 1. Standard curve of the protein Ct values of Hela cells versus the logarithm of the protein (ng). The cycle number at the threshold (Ct value) for each sample is interpolated onto the curve to calculate the relative activity of telomerase (RTA).

FIG. 2. Q-TRAP results bar chart (RTA) for in vitro treatments in primary fibroblast cultures. Data were pooled by condition and time of treatment.

FIG. 3. Growth curves of untreated adult primary human fibroblasts (control-DMSO) and treated with IG (IG_1, IG_2, IG_3) under standard conditions. Each point on the population curve represents the average of the triplicates for each cell passage.

FIG. 4. Growth curves of untreated adult primary human fibroblasts (control-MSO) and treated IG (IG_1, IG_2, IG_3) under oxidative conditions (H2O2 10 μM). Each point on the population curve represents the average triplicate for each cell step.

FIG. 5. The histogram shows the distribution of telomere lengths in a representative sample. The bars represent the relative frequency for each particular normalized fluorescence intensity (X-axis). The 20th percentile (red bars) indicates the particular length below which 20% of telomeres have been observed. The median (MTL) and mean length (ATL) of telomeres are also indicated in the histogram. This histogram also allows the analysis of telomere length variability.

FIG. 6. Both the DAPI (blue) and the fluorescence marked for the telomeric spots (pink & white) within each nucleus can be seen in the image.

FIG. 7. Bar graphs of TAT results. FIG. 7.a: Median telomere length. FIG. 7.b: 20th percentile. FIG. 7.c: % of telomeres of <3 kbp.

FIG. 8. Bar graphs of TAT results. FIG. 8.a: Median telomere length. FIG. 8.b: 20th percentile. FIG. 8.c: % of telomeres of <3 kbp, for the different treatments.

FIG. 9. Bar graphs for the telomere shortening ratio for different times and treatments under standard conditions.

FIG. 10. Bar graphs of shortening of the telomere ratio for the different treatments and times under conditions of oxidative stress.

FIG. 11 is a graph showing the effect of the composition in accordance with the invention at low doses on the longevity of C. elegans (N2).

FIG. 12 is a graph showing the effect of the composition in accordance with the invention in intermediate doses on the longevity of C. elegans (N2).

FIG. 13 is a graph showing the effect of the composition in accordance with the invention at high doses on the longevity of C. elegans (N2).

FIG. 14 shows survival curves of C. elegans (N2) obtained in populations treated with the composition in accordance with the invention (1 mg/mL) by the computer program GraphPad Prism.

FIG. 15 shows the average mobility of populations of C. elegans treated with the composition in accordance with the invention at different doses.

FIG. 16 shows the average mobility of populations of C. elegans treated with the composition in accordance with the invention at doses 0.1; 0.5 and 1 mg/mL.

FIG. 17 shows the frequency of curvatures of populations of C. elegans treated with the composition in accordance with the invention at doses 0.1; 0.5 and 1 mg/mL.

FIG. 18 shows the degree of dispersion of worms C. elegans treated with the composition in accordance with the invention at doses 0.1; 0.5 and 1 mg/mL.

FIG. 19 shows the antioxidant activity of the composition according to the invention evaluated at different doses (10-400 μg/mL) in C. elegans N2. The percentages of survival after applying oxidative stress with H₂O₂ are shown. Vitamin C (10 μg/mL) was included as a positive control. The data are for a single trial.

FIG. 20 shows the antioxidant activity of the composition in accordance with the invention evaluated at different doses in C. elegans N2. The percentages of survival after applying oxidative stress with H₂O₂ are shown. Vitamin C (10 μg/mL) was included as a positive control. Data are for the mean of 2 independent trials.

FIG. 21 represents the relative percentage increase in survival of C. elegans with the effective dose of the composition in accordance with the invention versus control condition, indicating an increase of 88.5%.

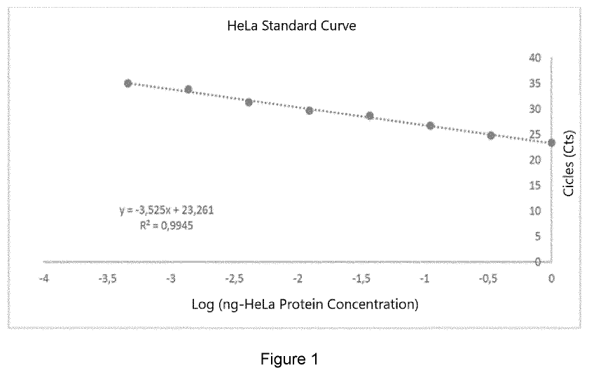

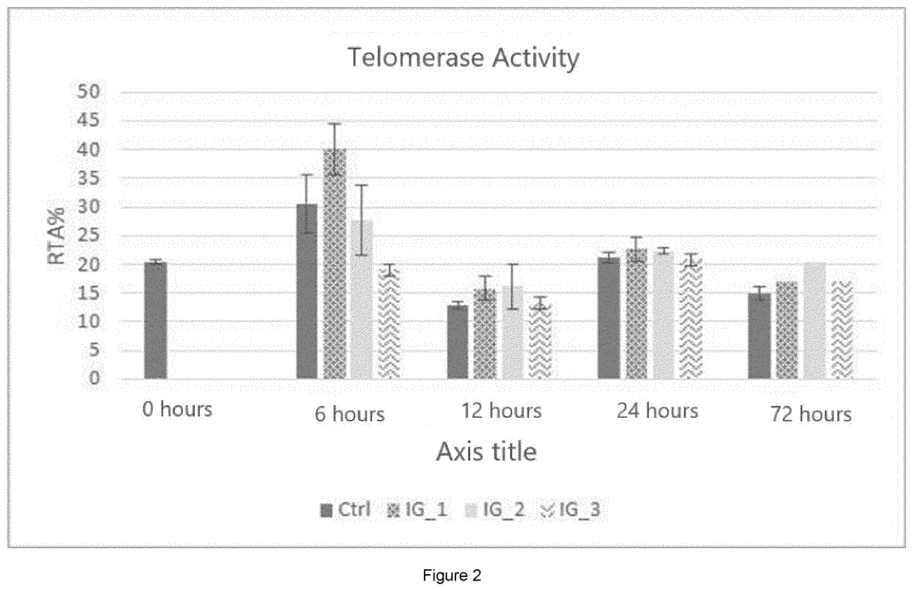

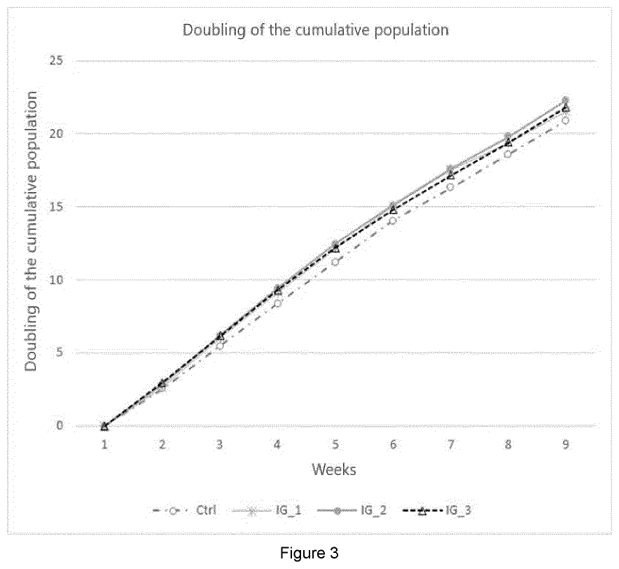

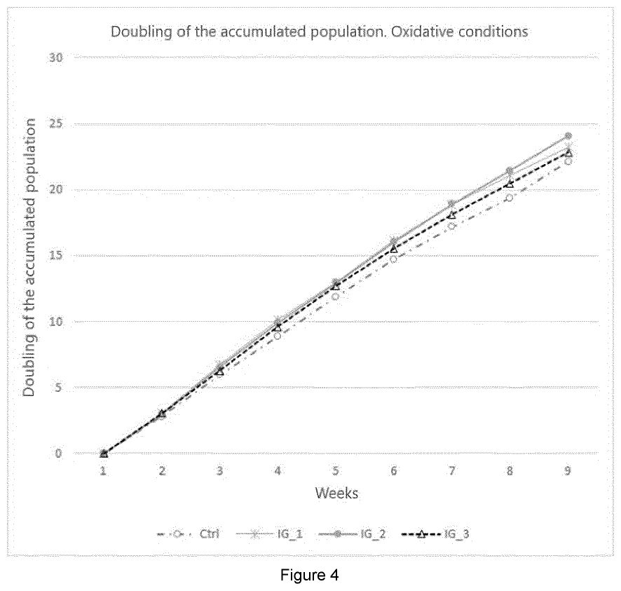

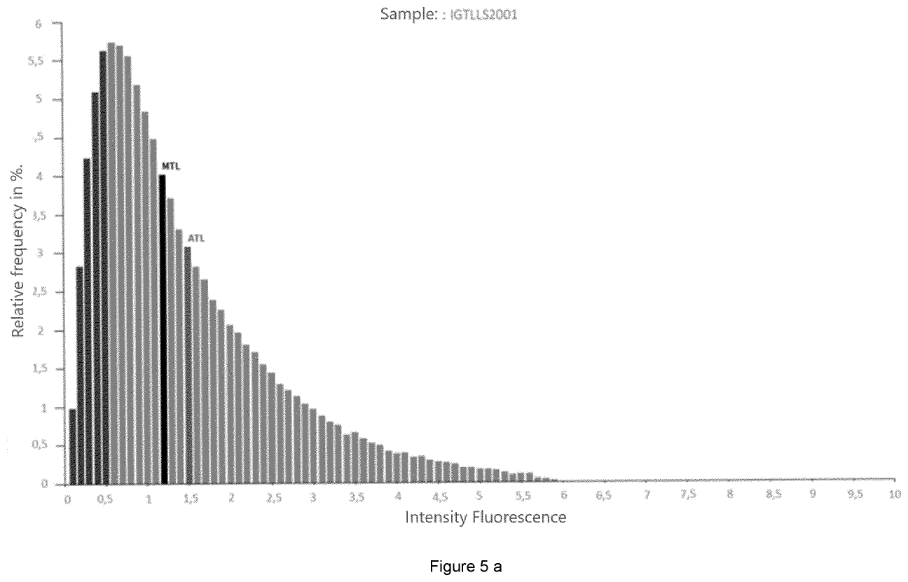

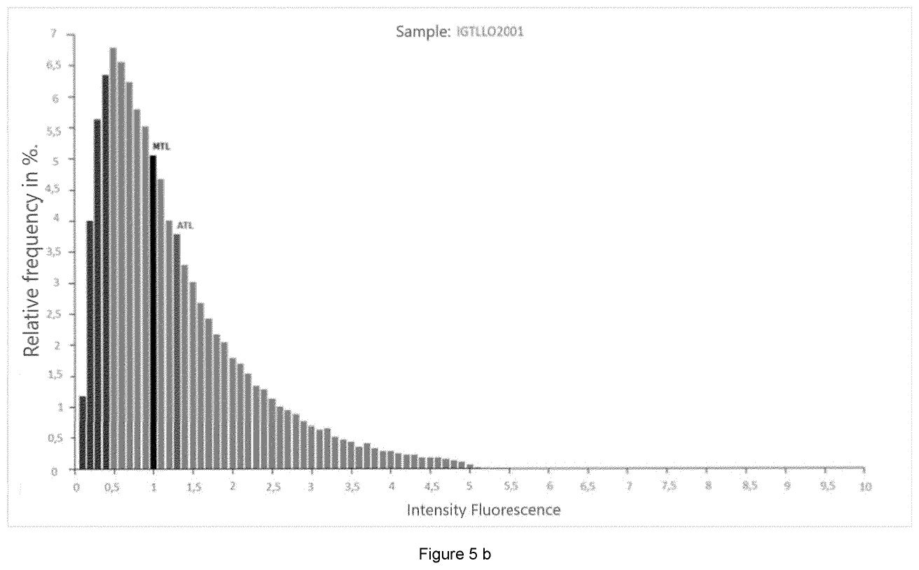

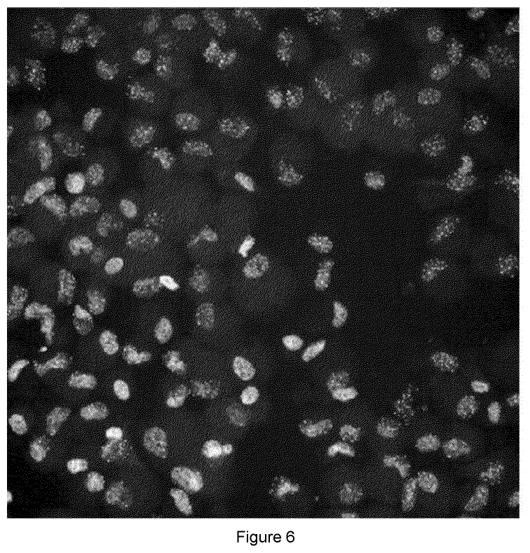

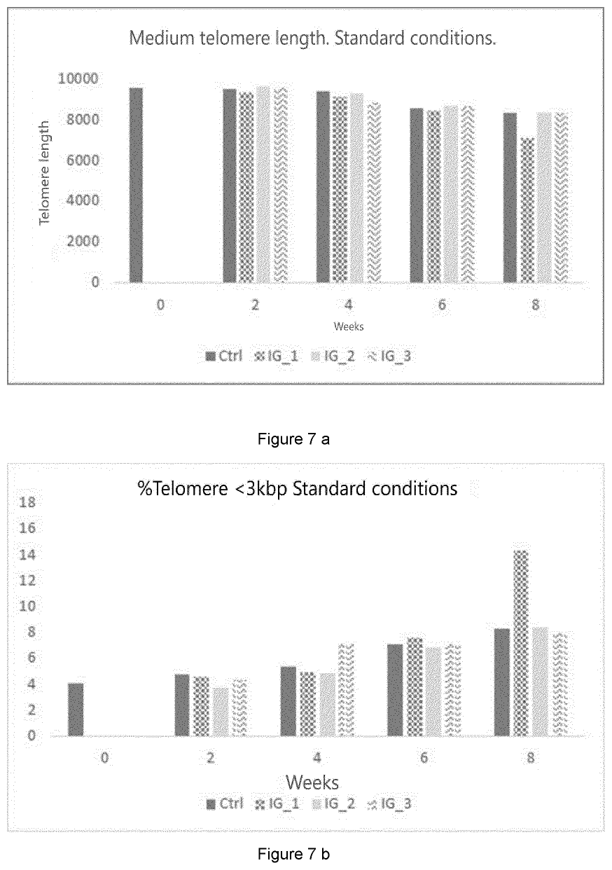

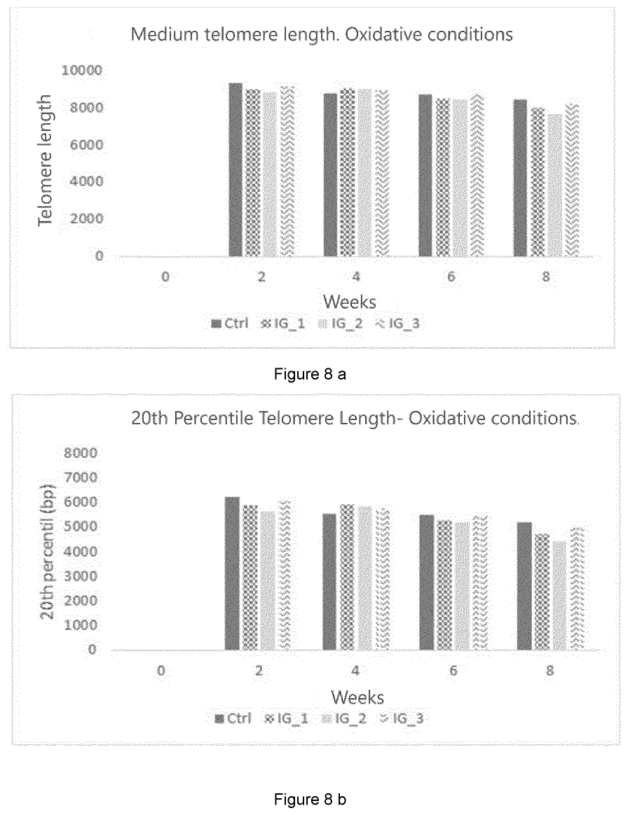

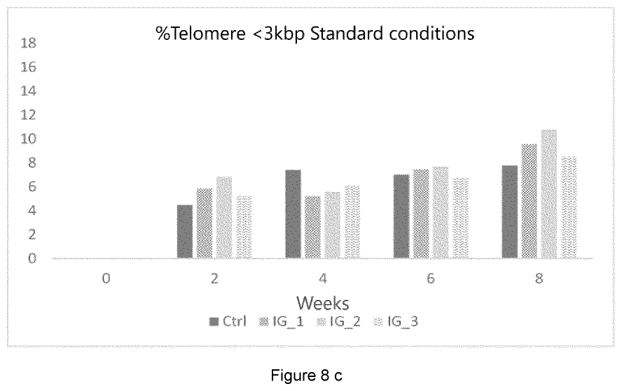

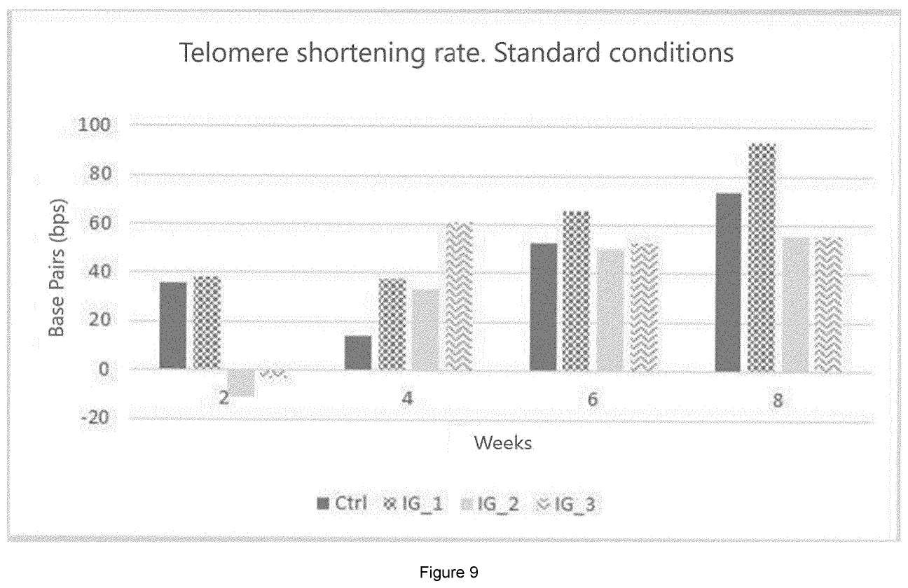

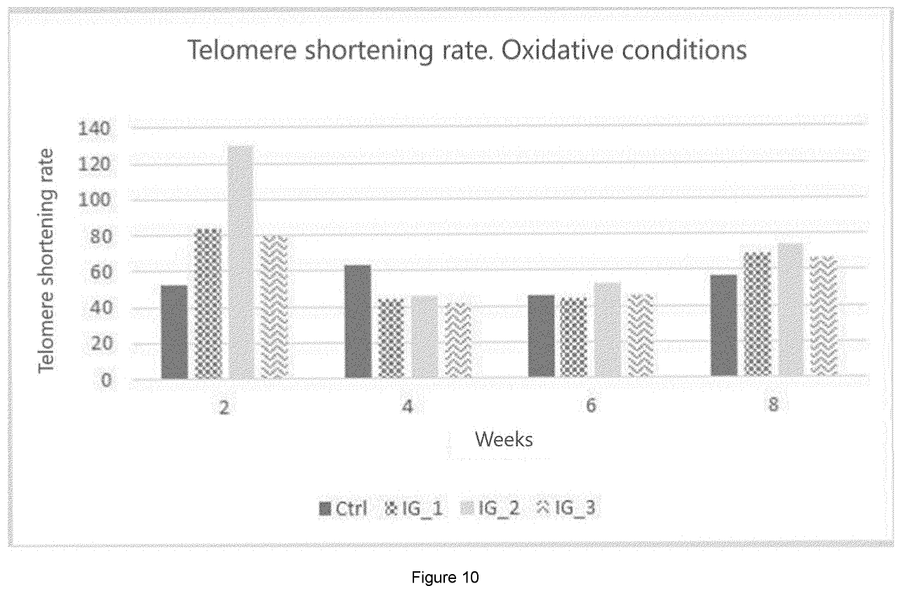

### DETAILED DESCRIPTION OF THE INVENTION

As indicated in the previous sections, the inventors have detected the need to develop a composition that is administered as an anti-aging agent, that is easy to administer, and that does not present toxicity.

Based on this, they have developed a composition that is administered orally, comprising lysates of microorganisms that is accompanied by other components for this composition, and that has turned out to be strikingly beneficial for humans and animals as it presents an antioxidant character and proves to be a promoter of cell proliferation, which makes it a highly effective anti-aging agent.

Thus, in a first aspect, the present invention relates to a composition of oral administration to prevent and delay aging, that it comprises lysates of probiotic microorganisms in the form of dry powder, in the following amount in percentage by weight with respect to the total:
5% to 30% of bacterial lysates of the genus Bacillus;
3% to 25% of bacterial lysates of the genus Bifidobacterium;
15% to 35% of bacterial lysates of the genus Lactobacillus;
30% to 65% of bacterial lysates of the genus Saccharomyces;
1.5% to 10% of bacterial lysates of the genus Streptococcus for use in the prevention and/or treatment of oxidative stress related with aging.

In accordance with the invention, the composition can comprise between 20%-99.5% by weight of bacterial lysates, particularly 25%-95% by weight of bacterial lysates, and more particularly 50%±10% by weight of bacterial lysates.

In the context of the present invention, bacterial lysates are understood as the product obtained after the process of cultivating, and subsequently, mechanically or chemically breaking said bacterial cells in order to obtain a product with microbial fragments, as well as all the components comprised therein. Likewise, in this document, it is indicated that these are dry bacterial lysates, since, thanks to their method of obtainment, these lysates are subsequently subjected to drying techniques, in such a way that said dry bacterial lysates are in powder form.

In preferred embodiments of the present invention, the bacterial lysates of the genus Bacillus are of the species that are selected from the group consisting of Bacillus clausii, Bacillus coagulans, Bacillus licheniformis, Bacillus pumilus, Bacillus mesentericus, Bacillus subtilis, and combinations thereof, or at least, Bacillus coagulans, Bacillus licheniformis, Bacillus mesentericus, Bacillus subtilis, and, optionally Bacillus clausii

In other embodiments, the bacterial lysates of the genus Bifidobacterium are of the species that are selected from the group consisting of Bifidobacterium animalis subsp lactis, Bifidobacterium bifidum, Bifidobacterium breve, Bifidobacterium lactis, Bifidobacterium longum, Bifidobacterium infantis, Bifidobacterium animalis, Bacillus paralicheniformis, and combinations thereof, or at least Bifidobacterium breve, and Bifidobacterium lactis and, optionally, Bifidobacterium bifidum.

In other embodiments, the bacterial lysates of the genus Lactobacillus are of the species that are selected from the group consisting of Lactobacillus acidophilus, Lactobacillus bulgaricus, Lactobacillus casei, Lactobacillus fermentum, Lactobacillus paracasei, Lactobacillus plantarum, Lactobacillus reuteri, Lactobacillus rhamnosus Lactobacillus salivarius, Lactobacillus helveticus, Lactobacillus gasseri, Lactobacillus johnsonii, Lactobacillus brevis, Lactobacillus kefiri, and combinations thereof or at least Lactobacillus acidophilus, Lactobacillus bulgaricus, Lactobacillus casei, Lactobacillus reuteri, and Lactobacillus rhamnosus, and, optionally, Lactobacillus fermentum.

In other particular embodiments of the present invention, the bacterial lysates of the genus Saccharomyces are selected from the group consisting of Saccharomyces cerevisiae, Saccharomyces boulardii, and combinations thereof.

In other particular embodiments of the present invention, bacterial lysates of the genus Streptococcus are of the species Streptococcus thermophilus, Streptococcus salivarius, and combinations thereof.

In preferred embodiments of the present invention, the composition object of the invention comprises bacterial lysates of the genus Bacillus in an amount in percentage by weight between 5% to 30%, preferably between 8% to 25%, and even more preferably between 10% to 18%. In preferred embodiments, the amount of bacterial lysates of the genus Bacillus can be 5%, 6%, 7%, 8%, 9% or 10%. In other preferred embodiments, the amount of bacterial lysates of the genus Bacillus can be 11%, 12%, 13%, 14%, 15%, 16%, 17%, 18%, 19%, 20%, 21%, 22%, 23%, 24%, 25%, 26%, 27%, 28%, 29% or 30%.

In preferred embodiments of the present invention, the composition object of the invention comprises bacterial lysates of the genus Bifidobacterium in an amount in percentage by weight between 3% to 25%, preferably between 5% to 20%, and even more preferably between 8% to 15%. In preferred embodiments, the amount of bacterial lysates of the genus Bifidobacterium can be 3%, 4% 5%, 6%, 7%, 8%, 9% or 10%. In other preferred embodiments, the amount of bacterial lysates of the genus Bifidobacterium can be 11%, 12%, 13%, 14%, 15%, 16%, 17%, 18%, 19%, 20%, 21%, 22%, 23%, 24% or 25%.

In preferred embodiments of the present invention, the composition object of the invention comprises bacterial lysates of the genus Lactobacillus in an amount in percentage by weight between 15% to 35%, preferably between 15% to 30%, and even more preferably between 20% to 25%. In preferred embodiments, the amount of bacterial lysates of the genus Lactobacillus can be 15%, 16%, 17%, 18%, 19% or 20%. In other preferred embodiments, the amount of bacterial lysates of the genus Lactobacillus can be 21%, 22%, 23%, 24%, 25%, 26%, 27%, 28%, 29%, 30%, 31%, 32%, 33%, 34% or 35%.

In preferred embodiments of the present invention, the composition comprises bacterial lysates of the genus Saccharomyces in an amount in percentage by weight between 30% to 65%, preferably between 50%-58%, and even more preferably between 35%-60%. In preferred embodiments, the amount of bacterial lysates of the genus Saccharomyces can be 30%, 31%, 32%, 33%, 34%, 35%, 36%, 37%, 38%, 39%, 40%, 41%, 42%, 43%, 44%, 45%, 46%, 47%, 48% or 49%. In other preferred embodiments, the amount of bacterial lysates of the genus Saccharomyces can be 50%, 51%, 52%, 53%, 54%, 55%, 56%, 57%, 58%, 59%, 60%, 61%, 62%, 63%, 64% or 65%.

In preferred embodiments of the present invention, the composition comprises bacterial lysates of the genus Streptococcus in an amount in percentage by weight between 1.5% to 10%, preferably between 2% to 7%, and even more preferably between 3% to 6%. In preferred embodiments, the amount of bacterial lysates of the genus Streptococcus can be 1.5%, 1.6%, 1.7%, 1.8%, 1.9%, 2%, 2.1%, 2.2%, 2.3%, 2.4%, 2.5%, 2.6%, 2.7%, 2.8%, 2.9% or 3%. In other preferred embodiments, the amount of bacterial lysates of the genus Streptococcus can be 3.5%, 4%, 4.5%, 5%, 5.5%, 6%, 6.5%, 7%, 7.5%, 8%, 8.5%, 9%, 9.5% or 10%.

In a particular embodiment of the invention, the composition for use, comprises lysates of probiotic microorganisms in an amount in percentage by weight of the total lysates of the composition:
approximately 10% to 25% of bacterial lysates of the genus Bacillus;
approximately 4% to 20% of bacterial lysates of the genus Bifidobacterium;
approximately 17% to 30% of bacterial lysates of the genus Lactobacillus;
approximately 35% to 60% of yeast lysates of the genus Saccharomyces;
approximately 2% to 8% of bacterial lysates of the genus Streptococcus.

This composition is called composition “A”

In a particular embodiment of the invention, the composition for use, comprises lysates of probiotic microorganisms in an amount in percentage by weight of the total lysates of the composition:
approximately 14% to 20% of bacterial lysates of the genus Bacillus;
approximately 6% to 15% of bacterial lysates of the genus Bifidobacterium;
approximately 18% to 25% of bacterial lysates of the genus Lactobacillus;
approximately 45% to 55% of yeast lysates of the genus Saccharomyces;
approximately 2% to 5% of bacterial lysates of the genus Streptococcus.

This composition is called composition “B”

In a particular embodiment of the invention, the composition for use, comprises lysates of probiotic microorganisms in an amount in percentage by weight of the total lysates of the composition:
16% of bacterial lysates of the genus Bacillus;
8% of bacterial lysates of the genus Bifidobacterium;
21% of bacterial lysates of the genus Lactobacillus;
52% of bacterial lysates of the genus Saccharomyces;
3% of bacterial lysates of the genus Streptococcus.

This composition is called composition “C”

In a particular embodiment of the invention, the composition for use, comprises lysates of probiotic microorganisms in an amount in percentage by weight of the total lysates of the composition:
5% to 30% of bacterial lysates of Bacillus coagulans, Bacillus licheniformis, Bacillus mesentericus, Bacillus subtilis, and, optionally Bacillus clausii;
3% to 25% of bacterial lysates of Bifidobacterium breve, and Bifidobacterium lactis and, optionally, Bifidobacterium bifidum;
15% to 35% of bacterial lysates of Lactobacillus acidophilus, Lactobacillus bulgaricus, Lactobacillus casei, Lactobacillus reuteri, and Lactobacillus rhamnosus, and, optionally, Lactobacillus fermentum;
30% to 65% of bacterial lysates of Saccharomyces cerevisiae;
1.5% to 10% of bacterial lysates of Streptococcus thermophilus

This composition is called composition “D”

In a particular embodiment of the invention, the composition for use, comprises lysates of probiotic microorganisms in an amount in percentage by weight of the total lysates of the composition:
10% to 25% of bacterial lysates of Bacillus coagulans, Bacillus licheniformis, Bacillus mesentericus, Bacillus subtilis, and, optionally Bacillus clausii;
4% to 20% of bacterial lysates of Bifidobacterium breve, and Bifidobacterium lactis and, optionally, Bifidobacterium bifidum;
17% to 30% of bacterial lysates of Lactobacillus acidophilus, Lactobacillus bulgaricus, Lactobacillus casei, Lactobacillus reuteri, and Lactobacillus rhamnosus, and, optionally, Lactobacillus fermentum;
35% to 60% of bacterial lysates of Saccharomyces cerevisiae;
2% to 8% of bacterial lysates of Streptococcus thermophilus

This composition is called composition “E”

In a particular embodiment of the invention, the composition for use, comprises lysates of probiotic microorganisms in an amount in percentage by weight of the total lysates of the composition:
14% to 20% of bacterial lysates of Bacillus coagulans, Bacillus licheniformis, Bacillus mesentericus, Bacillus subtilis, and, optionally Bacillus clausii;
6% to 15% of bacterial lysates of Bifidobacterium breve, and Bifidobacterium lactis and, optionally, Bifidobacterium bifidum;
18% to 25% of bacterial lysates of Lactobacillus acidophilus, Lactobacillus bulgaricus, Lactobacillus casei, Lactobacillus reuteri, and Lactobacillus rhamnosus, and, optionally, Lactobacillus fermentum;
45% to 55% of bacterial lysates of Saccharomyces cerevisiae;
2% to 5% of bacterial lysates of Streptococcus thermophilus

This composition is called composition “F”

In a particular embodiment of the invention, the composition for use, comprises lysates of probiotic microorganisms in an amount in percentage by weight of the total lysates of the composition:
Bacillus coagulans 0.1% to 10%; Bacillus licheniformis 1% to 12%; Bacillus mesentericus 1% to 12%; Bacillus subtilis 0.1% to 10%; and, optionally Bacillus clausii 0.1% to 5%;
Bifidobacterium breve 0.1% to 12%; and Bifidobacterium lactis 0.1% to 10%; and, optionally, Bifidobacterium bifidum 0.5% to 8%;
Lactobacillus acidophilus 0.3% to 15%; Lactobacillus bulgaricus 0.3% to 15%; Lactobacillus casei 0.3% to 12%; Lactobacillus reuteri 0.5% to 10%; and Lactobacillus rhamnosus 0.1% to 10%; and, optionally, Lactobacillus fermentum 0.5% to 10%;
Saccharomyces cerevisiae 35% to 60%;
Streptococcus thermophilus 2% to 8%;

This composition is called composition “G”

In a particular embodiment of the invention, the composition for use, comprises lysates of probiotic microorganisms in an amount in percentage by weight of the total lysates of the composition:
Bacillus coagulans 0.1% to 6%; Bacillus licheniformis 2% to 10%; Bacillus mesentericus 2% to 10%; Bacillus subtilis 0.3% to 8%; and, optionally Bacillus clausii 0.1% to 4%;
Bifidobacterium breve 1% to 10%; and Bifidobacterium lactis 1% to 8%; and, optionally, Bifidobacterium bifidum 1% to 6%;
Lactobacillus acidophilus 1% to 12%; Lactobacillus bulgaricus 1% to 12%; Lactobacillus casei 1% to 10%; Lactobacillus reuteri 1% to 7%; and Lactobacillus rhamnosus 0.3% to 7%; and, optionally, Lactobacillus fermentum 1% to 7%;
Saccharomyces cerevisiae 45% to 55%;
Streptococcus thermophilus 2% to 5%;

This composition is called composition “H”

In a particular embodiment of the invention, the composition for use, comprises lysates of probiotic microorganisms in an amount in percentage by weight of the total lysates of the composition:
Bacillus coagulans 0.1% to 4%; Bacillus licheniformis 3% to 8%; Bacillus mesentericus 3% to 8%; Bacillus subtilis 0.1% to 10%; and, optionally Bacillus clausii 0.5% to 7%;
Bifidobacterium breve 1.5% to 8%; and Bifidobacterium lactis 1.5% to 5%; and, optionally, Bifidobacterium bifidum 1.5% to 4%;
Lactobacillus acidophilus 2% to 10%; Lactobacillus bulgaricus 2% to 10%; Lactobacillus casei 2% to 8%; Lactobacillus reuteri 1.5% to 5%; and Lactobacillus rhamnosus 0.5% to 5%; and, optionally, Lactobacillus fermentum 2% to 5%;
Saccharomyces cerevisiae 47% to 56%;
Streptococcus thermophilus 2% to 4%;

This composition is called composition “I”

In a particular embodiment of the invention, the composition for use, comprises the lysates of probiotic microorganisms in the form of dry powder, in the amount as a percentage by weight of the total as disclosed in compositions “A”, “B”, “C”, “D” E”, “F”, “G”, “H” or “I”.

In other particular embodiments of the present invention, the composition object of the invention may comprise another additional component or additive that is selected from the group consisting of Riboflavin, Vitamin C, Manganese, Selenium, Zinc, Vitamin D and Magnesium.

Riboflavin, Vitamin C, Magnesium, Selenium and Zinc contribute to the protection of cells against oxidative damage. Vitamin D, Manganese and Zinc contribute to the maintenance of normal bones. Vitamin D and Magnesium contribute to the normal formation of collagen for the normal functioning of bones. Vitamin D, Vitamin C, Zinc and Selenium contribute to the normal functioning of the immune system.

Next, we indicate the quantity in which could be included each of the additional components or additives that can optionally comprise the composition, in accordance with the first aspect of the present invention.

TABLE 1
List of the various additional components that the composition
object of the invention can comprise, and the quantities
in which these components are found in each of the particular
and preferred embodiments of the present invention.

| | | | |
|---|---|---|---|
| | | | In even more |
| | In particular | In preferred | preferred |
| Additional | embodiments | embodiments | embodiments |
| components | (Quantity) | (Quantity) | (Quantity) |

| | | | | | | |
|---|---|---|---|---|---|---|
| Vitamin C | 10-150 | mg | 50-100 | mg | 72 | mg |
| Vitamin D | 0.5-10 | μg | 1-8 | μg | 4.5 | μg |
| Riboflavin | 0.01-2 | mg | 0.5-1 | mg | 0.99 | mg |
| Magnesium | 25-100 | mg | 50-90 | mg | 75 | mg |
| Manganese | 0.1-2 | mg | 1.1-2 | mg | 1.8 | mg |
| Zinc | 0.5-40 | mg | 1-30 | mg | 9 | mg |
| Selenium | 10-300 | μg | 20-150 | μg | 49.5 | μg |

In particular embodiments of the present invention, the composition object of the invention, are obtained by a method comprising the following steps:
Cultivate the microbial strains selected for the preparation of the composition using the standard conditions established for the microbial species listed in this document;
Filter or centrifuge the cultures until a biomass of the selected bacterial lysates is obtained;
Lyse by freezing and thawing cycles, and sonication of the biomasses obtained until securing at least 90% of bacterial lysates, preferably at least 95%, and more preferably 99%;
Dry by atomization or lyophilization the biomasses of bacterial lysates obtaining a product in powder form comprising all the bacterial strains selected to prepare the composition object of the invention.

Thus, the composition object of the invention is presented in powder form.

Based on the fact that the composition object of the invention is in powder form, the composition, in accordance with the first aspect of the invention, can be presented in sealed sachets, in themselves conventional in the food and pharmaceutical industry.

Another form of presentation of the food supplement, in accordance with the invention, is in capsule form, such as conventional gelatin capsules, inside which the composition in powder form is contained.

In a further aspect, the invention relates to the use of the composition object of the invention as a food supplement for the prevention and delay of aging in animal or human cells.

In a further aspect, the invention relates to the composition defined herein for use as a medicament.

In a further aspect, the invention relates to the composition that is defined herein for its use as an anti-aging agent.

In a further aspect, the invention relates to a pharmaceutical composition comprising an effective pharmaceutical amount of the composition in accordance with the first aspect of the invention and a pharmaceutically acceptable excipient.

In the context of the present invention, what is known as an anti-aging agent, which can also be called an anti-maturing agent, is a compound that has an ability to fight aging, whose effect it is to delay the effect of aging and the proliferative capacity of cells.

In the context of the present invention, the expression “pharmaceutical composition” refers to a formulation that has been adapted to deliver a predetermined dose of one or more useful therapeutic agents to a cell, a group of cells, an organ or a tissue.

The term “effective pharmaceutical quantity”, as used herein, is understood as an amount capable of providing a therapeutic effect, and which can be determined by the person skilled in the art by commonly used means.

Also, in the context of the present invention, “pharmaceutically acceptable excipient” means a therapeutically inactive substance that is said to be used to incorporate the active ingredient and that is acceptable to the patient from a pharmacological/toxicological standpoint and to the pharmaceutical chemist who manufactures it from a physical/chemical standpoint, with respect to composition, formulation, stability, patient acceptance and bioavailability.

In the context of the present invention, a food supplement is understood as a food product intended to supplement the normal diet and consisting of concentrated sources of nutrients or other substances that have a nutritional or physiological effect, in single or combined form, is marketed in dosed form, i.e. capsules, pastilles, tablets, pills and other similar forms, sachets of powders, ampoules of liquid, bottles with droppers and other similar forms of liquids and powders to be taken in small unit quantities, as defined in Directive 2002/46/EC of the European Parliament.

In a particular embodiment of the present invention, the use of the composition as a food supplement comprises orally administering an amount between 20 mg to 200 mg of the composition, 2 to 6 times a day. As such, the use of the composition as an anti-aging agent is indeed the object of the invention, particularly prevention and/or treatment of oxidative stress related with aging

In a particular embodiment of the present invention, the use of the composition as a food supplement comprises orally administering to an individual a dose at least twice a day. Also, for the treatment to be effective and to achieve the desired effect, this dose should be administered to the patient at least twice a day and a maximum of 6 times a day. Preferably, the administration of the dose to the patient is 3, 4 or 5 times a day, most preferably 3 times a day.

In the context of the present invention, a dose is defined as the amount of medicine containing the exact measure of active ingredient so as to be efficient, effective and safe for the patient, and that solves the health problem for which it has been indicated.

Thus, in the context of the present invention, it has been determined that a dose has an amount of the composition object of the invention of between 20 mg to 200 mg. In preferred embodiments, a dose can comprise between 80 mg to 150 mg of the composition. In particular embodiments of the present invention, a dose may comprise 20 mg, 30 mg, 40 mg, 50 mg, 60 mg, 70 mg, 80 mg, 90 mg, 20 mg, 100 mg, 110 mg, 120 mg, 130 mg, 140 mg, 150 mg, 160 mg, 170 mg, 180 mg, 190 mg or 200 mg of the composition object of the invention.

In another particular embodiment, it has been determined that a dose has an amount of the composition object of the invention of between 1 μg to 20 mg. In preferred embodiments, a dose can comprise between 5 μg to 20 mg of the composition. In particular embodiments of the present invention, a dose may comprise 20 μg, 30 μg, 40 μg, 50 μg, 60 μg, 70 μg, 80 μg, 90 μg, 20 μg, 100 μg, 110 μg, 120 μg, 130 μg, 140 μg, 150 μg, 160 μg, 170 μg, 180 μg, 190 μg or 200 μg, 500 μg, 1 mg, 2 mg, 3 mg, 4 mg, 5 mg, 6 mg, 7 mg, 8 mg, 9 mg, or 10 mg of the composition object of the invention.

In another particular embodiment, the composition for use comprises administering to a subject an effective amount of the composition. The amount of compound that prevents and/or treats of oxidative stress related with aging can be an amount from a low of about 0.1 μg/ml, about 0.2 μg/ml, or about 0.5 μg/ml, to a high of about 100 μg/ml, about 200 μg/ml, or about 500 μg/ml. For example, the amount of compound that delays aging can be from about 0.1 μg/ml to about 100 μg/ml, from about 0.2 μg/ml to about 200 μg/ml, from about 0.5 μg/ml to about 500 μg/ml, from about 0.1 μg/ml to about 10 μg/ml, from about 0.1 μg/ml to about 20 μg/ml, from about 0.1 μg/ml to about 50 μg/ml, from about 0.5 μg/ml to about 20 μg/ml, from about 0.5 μg/ml to about 50, from about 0.5 μg/ml to about 100 μg/ml, from about 0.5 to about 200 μg/ml, from about 0.5 g/ml to about 500 μg/ml, from about 15 μg/ml to about 25 μg/ml, from about 15 μg/ml to about 50, from about 18 μg/ml to about 30 μg/ml, from about 18 μg/ml to about 50 μg/ml, or from about 1 μg/ml to 10 μg/ml, or 2 μg/ml to 8 μg/ml or 10 μg/ml to 30 mg/ml or 50 μg/ml to 30 mg/ml. These amounts can be daily, once per week, once per more, or from 2 to 6 times a day. For example, a dose between 50 μg/ml to 30 mg/ml daily.

The term “preventing” as used herein refers to administering a compound prior to the onset of clinical symptoms of a disease or conditions so as to prevent a physical manifestation of aberrations associated with the disease or condition. In the context of oxidative stress associated with aging, the term “preventing” refers to administering a compound prior to the onset of clinical symptoms of oxidative stress associated with aging so as to prevent a physical manifestation of aberrations associated with oxidative stress related with aging.

The terms “treatment” and “treating” as used herein refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. In the context of a subject suffering from oxidative stress associated with aging, the terms “treatment” and “treating” refer to the medical management of a subject with the intent to cure, ameliorate, or stabilize oxidative stress related with aging. In the context of a subject at risk of developing oxidative stress associated with aging, the terms “treatment” and “treating” refer to the medical management of a subject with the intent to prevent oxidative stress related with aging.

In particular embodiments where the composition is presented in watertight sachets, this composition can be administered to the patient dissolved or suspended in a liquid, preferably in an aqueous liquid, and most preferably in beverages such as fruit juices, milk, water. In addition, it can also be mixed with foods such as yogurt, liquid yogurt, soups, purees, creams, or porridge. These foodstuffs have to be at optimum temperature to be consumed and must never be heated after having added the composition object of the invention.

The composition may comprise an abundance relative to all microbial proteins of at least 85% of proteins of Saccharomyces culture lysates. Equally, the composition may comprise a relative abundance of at least 5-0.02% of proteins from culture lysates of each of the following microorganisms: Bacillus, Lactobacillus, Streptococcus, Saccharomyces, and Bifidobacterium.

The functional analysis based on the KEGG Orthology (KO) carried out to check the functions of proteins in the different metabolic pathways that are defined by KEGG and COG, revealed as noteworthy functions in the protein profile K03530, which are histone-like proteins with DNA binding function in a relative abundance of 0.5-3%. In preferred embodiments, it is found in a relative abundance of 0.6% to 2%, more preferably of 0.7% to 1%, and even more preferably of 0.7%. These are proteins that play an important role in epigenetic regulation with various covalent combinations. Total histone protein levels decline during aging and overexpression dramatically prolongs lifespan and promotes host health.

The KO categories correspond to the classification of the Kyoto Encyclopedia of Genes and Genomes (=KEGG Orthologies; KEGG=Kyoto Encyclopedia of Genes and Genomes-https://www.genome.jp/kegg/).

In preferred embodiments of the present invention, the composition may comprise an abundance relative to the totality of microbial proteins between 1.50 to 9% of proteins from bacterial lysates of the genus Bacillus in percentage by weight, preferably between 2% to 7%, and even more preferably between 4.45% to 6.35%. In preferred embodiments, the relative abundance of proteins from bacterial lysates of the genus Bacillus can be 2%, 2.50%, 3%, 3.50%, 4%, 4.50%, 5%, 5.50%, 6%, 6.50%, 7%, 7.50%, 8%, 8.5% or 9%.

In preferred embodiments of the present invention, the proteins that have been identified from bacterial lysates of the genus Bacillus are selected from the group consisting of Q65HF3, P54944, Q5WEC7, and combinations thereof.

In preferred embodiments of the present invention, the composition may comprise an abundance relative to the totality of microbial proteins between 0.01% to 1.20% of proteins from bacterial lysates of the genus Bifidobacterium in percentage by weight, and preferably between 0.07% to 1.10%. In preferred embodiments, the relative abundance of proteins from bacterial lysates of the genus Bifidobacterium can be 0.01%, 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 0.95%, 1.10%, 1.15% or 1.20%.

In preferred embodiments of the present invention, the proteins that have been identified from bacterial lysates of the genus Bifidobacterium are selected from the group consisting of B8DTX9, B8DSQ4, and combinations thereof.

In preferred embodiments of the present invention, the composition may comprise an abundance relative to the totality of microbial proteins of at least 2% to 8% of proteins from bacterial lysates of the genus Lactobacillus in an amount in percentage by weight preferably between 3.40% to 7.55%, and even more preferably between 4% to 5.45%.

In preferred embodiments, the relative abundance of proteins from bacterial lysates of the genus Lactobacillus can be 2%, 2.50%, 3%, 3.50%, 4%, 4.50%, 5%, 5.50%, 6%, 6.50%, 7%, 7.50% or 8%.

In preferred embodiments of the present invention, the proteins that have been identified from bacterial lysates of the genus Lactobacillus are selected from the group consisting of P35829, A0A0H0YNJ3, A5VJ92, Q1G910, and combinations thereof.

In preferred embodiments of the present invention, the composition may comprise an abundance relative to the totality of microbial proteins between 49% to 99.00% of proteins from bacterial lysates of the genus Saccharomyces in an amount in percentage by weight preferably between 65% to 90%, and even more preferably between 75% to 85%. In preferred embodiments, the relative abundance of proteins from bacterial lysates of the genus Saccharomyces can be 49%, 50%, 51%, 52%, 53%, 54%, 55%, 56%, 57%, 58%, 59%, 60%, 61%, 62%, 63%, 64%, 65%, 66%, 67%, 68%, 69% or 70%. In other preferred embodiments, the relative abundance of proteins from bacterial lysates of the genus Saccharomyces can be 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

In preferred embodiments of the present invention, the proteins that have been identified from bacterial lysates of the genus Saccharomyces are selected from the group consisting of E7NI79, E7LV64, AOAOL8VTA7, E7Q2B5, E7NEQ3, A0A0L8VV91, A0A0L8VIG1, A0A0L8VIS1, and combinations thereof.

In preferred embodiments of the present invention, the composition may comprise an abundance relative to the totality of microbial proteins between 0.15% to 0.90% of proteins from bacterial lysates of the genus Streptococcus in percentage by weight, preferably between 0.17% to 89%, and even more preferably between 0.33% to 66%. In preferred embodiments, the relative abundance of proteins from bacterial lysates of the genus Streptococcus can be 0.15%, 0.20%, 0.25%, 0.30%, 0.35%, 0.40%, 0.45%, 0.50%, 0.55%, 0.60%, 0.65%, 070%, 0.75%, 0.80%, 0.85% or 0.90%.

In preferred embodiments of the present invention, the proteins that have been identified from bacterial lysates of the genus Streptococcus are selected from the group consisting of Q5M5J3, Q5M2M7, and combinations thereof.

In particular embodiments, the composition object of the invention is obtained from cultures of probiotic microorganism of the microorganisms of the genera described herein, which comprise a certain amount of Colony Forming Units (in English: CFU). In the context of the present invention, a Colony Forming Unit is a term of microbiology. It is an indicator of the amount of live microorganisms present in a medium.

In particular embodiments of the present invention, one starts from a number of bacteria of the genus Bacillus comprising between 3.49% to 20.94% of CFU with respect to the total CFU of the composition, most preferably between 10.98% to 15.75%.

In particular embodiments of the present invention, one starts from a number of bacteria of the genus Lactobacillus comprising between 28.64% to 66.82% of CFU with respect to the total CFU of the composition, most preferably between 40.66% to 57.36%.

In particular embodiments of the present invention, one starts from a number of bacteria of the genus Streptococcus comprising between 5.72% to 38.15% of CFU with respect to the total CFU of the composition, most preferably between 10.98% to 20.75%.

In particular embodiments of the present invention, one starts from a number of bacteria of the genus Saccharomyces comprising between 11.44% to 24.7 9% of CFU with respect to the total culture, most preferably between 19.04% to 21.75%.

In particular embodiments of the present invention, one starts from a number of bacteria of the genus Bifidobacterium comprising between 10.73% to 53.64% of CFU with respect to the total culture, most preferably between 15.9 9% to 18.75%.

These probiotic bacteria are cultured under standard conditions, as set out in the culture protocols published by Colection Española de Cultivos Tipo (CECT), indicated for each of the bacterial species described in this document.

Once cultured, these microorganisms are subjected to a lysis procedure. The process of obtaining lysates consists in combining a non-mechanical method with a mechanical one. First of all, the microbial cells undergo heat treatment. Each batch of viable cell culture undergoes a sterilization cycle in an autoclave at 121° C. for 20 to 30 minutes.

This temperature denatures and coagulates the proteins by inactivating them. It also causes membrane damage, ribosome aggregation, DNA strand breakdown and enzyme inactivation. Once cold, they undergo a cell rupture treatment by sonication for a period of 15 to 20 min, at an output power of between 450 to 550 W, at an amplitude of between 38 to 43% and a period of 8 to 15 seconds of pause, obtaining a mass of lysed cells from the probiotic batch (Qsonica, Q500). One obtains breakage of intermolecular interactions and DNA fragmentation. The final solution is freeze-dried and ground to obtain a lysate of the powdery probiotic microorganism. The powder is kept in a cool environment away from heat.

The composition, in accordance with the invention, is the result of the combination of probiotic bacteria that after a process of growth and lysis generates an extract consisting of a set of metabolites, proteins, DNA fragments and other components, such as, for example, peptidoglycans, which dosed efficiently is able to alter and modify in a striking way the host microbiota through several mechanisms, so as to confer an activation of the immune system reversing dysbiosis and strikingly enhancing the delay of cellular aging.

In a further aspect, the invention relates to the use of a composition of oral administration to prevent and delay aging, that comprises lysates of probiotic microorganisms in the form of dry powder, in the following amount in percentage by weight with respect to the total:
5% to 30% of bacterial lysates of the genus Bacillus;
3% to 25% of bacterial lysates of the genus Bifidobacterium;
15% to 35% of bacterial lysates of the genus Lactobacillus;
30% to 65% of bacterial lysates of the genus Saccharomyces;
1.5% to 10% of bacterial lysates of the genus Streptococcus;

In a particular embodiment the use of the composition is to prevent and delay aging in animal or human cells.

In another particular embodiment of the invention, the composition comprises the lysates of probiotic microorganisms in the form of dry powder, in the amount as a percentage by weight of the total as disclosed in compositions “A”, “B”, “C”, “D” E”, “F”, “G”, “H” or “I”.

In another particular embodiment, the use comprises administering the composition in an amount between 20 mg to 200 mg of the composition and 2 to 6 times a day orally.

In another particular embodiment, the use comprises administering to a subject an effective amount of the composition. The amount of compound that delays aging can be an amount from a low of about 0.1 μg/ml, about 0.2 μg/ml, or about 0.5 μg/ml, to a high of about 100 μg/ml, about 200 μg/ml, or about 500 μg/ml. For example, the amount of compound that delays aging can be from about 0.1 μg/ml to about 100 μg/ml, from about 0.2 μg/ml to about 200 μg/ml, from about 0.5 μg/ml to about 500 μg/ml, from about 0.1 μg/ml to about 10 μg/ml, from about 0.1 μg/ml to about 20 μg/ml, from about 0.1 μg/ml to about 50 μg/ml, from about 0.5 μg/ml to about 20 μg/ml, from about 0.5 μg/ml to about 50, from about 0.5 μg/ml to about 100 μg/ml, from about 0.5 to about 200 μg/ml, from about 0.5 μg/ml to about 500 μg/ml, from about 15 μg/ml to about 25 μg/ml, from about 15 μg/ml to about 50, from about 18 μg/ml to about 30 μg/ml, from about 18 μg/ml to about 50 μg/ml, or from about 1 μg/ml to 10 μg/ml, or 2 μg/ml to 8 μg/ml or 10 μg/ml to 30 mg/ml or 50 μg/ml to 30 mg/ml. These amounts can be daily, once per week, once per more, or from 2 to 6 times a day. For example, a dose between 50 μg/ml to 30 mg/ml daily.

The technical effect derived from the composition object of the invention is that it acts directly on the microbiota in humans. Thanks to this restorative and modulating action, the inventors have confirmed that, strikingly, intervening in the microbiota of human beings can significantly improve the state of health of people, and in particular, slow down the aging process, which materializes in delay and prevention of the development of decreased energy, the change in appearance in humans and animals due to such aging, as demonstrated by the experimental results described herein.

In this way the inventors have been able to verify that the oral administration of the composition object of the invention exerts an effect on the adjustment and reprogramming of the microbiota present in the intestinal tract of people, and that it is not just a local effect since the whole organism benefits from this modulation of the microbiota, thus becoming a systemic effect.

The invention also relates to the following clauses
1. A composition of oral administration to prevent and delay aging, characterized in comprising lysates of probiotic microorganisms in the form of dry powder, in the following amount as a percentage by weight of the total:
   5% to 30% of bacterial lysates of the genus Bacillus;
   3% to 25% of bacterial lysates of the genus Bifidobacterium;
   15% to 35% of bacterial lysates of the genus Lactobacillus;
   30% to 65% of bacterial lysates of the genus Saccharomyces;
   1.5% to 10% of bacterial lysates of the genus Streptococcus.
2. The composition in accordance with clause 1, wherein the bacterial lysates of the genus Bacillus are of the species that are selected from the group consisting of Bacillus clausii, Bacillus coagulans, Bacillus licheniformis, Bacillus pumilus, Bacillus subtilis, Bacillus paralicheniformis, and combinations thereof.
3. The composition in accordance with clauses 1 or 2, wherein the bacterial lysates of the genus Bifidobacterium are of the species that are selected from the group consisting of Bifidobacterium animalis subsp lactis, Bifidobacterium bifidum, Bifidobacterium breve, Bifidobacterium lactis, Bifidobacterium longum, Bifidobacterium infantis, Bifidobacterium animalis, and combinations thereof.
4. The composition in accordance with any of clauses 1 to 3, wherein the bacterial lysates of the genus Lactobacillus are of the species that are selected from the group consisting of Lactobacillus acidophilus, Lactobacillus bulgaricus, Lactobacillus casei, Lactobacillus fermentum, Lactobacillus paracasei, Lactobacillus plantarum, Lactobacillus reuteri, Lactobacillus rhamnosus Lactobacillus salivarius, Lactobacillus helveticus, Lactobacillus gasseri, Lactobacillus johnsonii, Lactobacillus brevis, Lactobacillus kefiri, and combinations thereof.
5. The composition in accordance with any of clauses 1 to 4, wherein the bacterial lysates of the genus Saccharomyces are selected from the group consisting of Saccharomyces cerevisiae, Saccharomyces boulardi, and combinations thereof.
6. The composition in accordance with any of clauses 1 to 5, wherein the bacterial lysates of the genus Streptococcus are of the species Streptococcus thermophilus, Streptococcus salivarius, and combinations thereof.
7. The composition in accordance with any of clauses 1 to 6, comprising at least one additional component that is selected from the group consisting of Riboflavin, Vitamin C, Manganese, Selenium, Zinc, Vitamin D and Magnesium.
8. The composition in accordance with any of clauses 1 to 7, wherein comprises a relative abundance, as a percentage by weight, comprised between:
   1.50 to 9% of proteins from bacterial lysates of the genus Bacillus;
   0.01% to 1.20% of proteins from bacterial lysates of the genus Bifidobacterium;
   2% to 8% of proteins from bacterial lysates of the genus Lactobacillus;
   49% to 99.00% of proteins from bacterial lysates of the genus Saccharomyces;
   0.15% to 0.90% of proteins from bacterial lysates of the genus Streptococcus.
9. The use of the composition in accordance with any of clauses 1 to 8 as a food supplement for the prevention and delay of aging in animal or human cells.
10. The use of the composition in accordance with clause 9, wherein the composition is administered an amount between 20 mg to 200 mg of the composition and 2 to 6 times a day orally.
11. The use of the composition in accordance with clauses 9 or 10, wherein the composition is presented in the form of dry powder inside watertight sachets or encapsulated in gelatin capsules.
12. The composition in accordance with any of clauses 1 to 8, for use as a medicament.
13. The composition in accordance with claim 12, for use in the prevention and delay of aging in animal or human cells.
14. The use of the composition in accordance with clauses 12 or 13, wherein the composition is administered an amount between 20 mg to 200 mg of the composition and 2 to 6 times a day orally.
15. A pharmaceutical composition comprising an effective pharmaceutical amount of the composition in accordance with any of claims 1 to 8, and a pharmaceutically acceptable excipient.

Therefore, the main advantages arising from the composition object of the invention are the following:
supplement and/or complement the physiological functions of the microbiota of humans and animals;
The alterations that generate dysbiosis and have an impact on oxidative and aging processes are restored;
the fact that the administration of the product is oral, makes the present composition and its striking effect a very advantageous alternative compared to anti-aging treatments currently used in medicine;
The present composition includes lysates of microorganisms, that is, it does not present living organisms such that this composition is presented as a safe alternative, since it would avoid the possible dangers of colonizing organisms, and also does not present the toxicity that a conventional drug can present.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%”, “80% to 95%” and “90% to 95%”). Certain implementations of the technology are set forth in examples below

### PREFERRED EMBODIMENT OF THE INVENTION

In order to contribute to a better understanding of the invention, and in accordance with a practical realization thereof, we have accompanied as an integral part of this description a preferred embodiment of the present invention.

### Example 1-Preparation of a Lysate Composition of Probiotic Microorganisms

The process consists in obtaining the mixture of dry microbial lysates of different strains of Bacillus sp., Lactobacillus sp., Bifidobacterium sp., Streptococcus sp. and Saccharomyces sp. To achieve this, these are grown by fermentation, concentrated, broken-up, and finally the cellular components are dried. The postbiotic formulation will be the mixture of lysates that can be accompanied by food ingredients in a capsule or powder supplement format.

For growth, an inoculum is prepared in a culture medium in a flask of each microorganism, in adequate quantity and volume to be able to proceed to inoculate the fermenter with respective incubated inoculums. To do this, the microorganisms preserved in vials with 20% glycerol at −80° C. are immersed in a water bath at 30° C. to allow a quick and complete thawing of the respective microorganisms.

Once the inocula of the respective microorganisms have been obtained, the fermenters are sown directly separately. The percentage of sowing is 3% for Bacillus species and 5% for each species of Bifidobacterium, Lactobacillus, Streptococcus and Saccharomyces.

After the sowing of the fermenter with the inoculum of the corresponding microorganism, we proceed to the fermentation phase of each of the microorganisms for the generation of microbial biomass, establishing the growth conditions for each species.

For the cultivation of Bacillus, the inoculum is grown in Nutrient Broth (NB) at 30° C. for 15 h at 250 rpm. It is cultured in a fermentation medium composed of Glucose H₂O 25 g/l, Yeast extract 5 g/l, K₂HPO₄ 2.5 g/l, MgSO₄·7H₂O 5 g/l, Tween 80 1 g/l, at 30° C.±1° C. throughout the process and pH 6.8±0.1 by sterile addition of NH₄OH 25% or H₃PO₄ 35% automatically. Dissolved oxygen is maintained ≥40% by stirring (150-250 rpm) and aeration (1-1.5vvm) with a working pressure of 0.5 atm. After approximately 20-24 h, the base consumption (NH₄OH) is stopped, and the temperature is lowered to 4-8° C. to stop cell metabolism and harvest the broth.

In the case of Streptococcus, the inoculum is grown in Tryptic Soy Broth (TSB) at 37° C. with a stirring of 150 rpm for 24 h. The fermentation medium consists of Glucose 25 g/l, Milk powder 8 g/l, Yeast extract 5 g/l, K₂HPO₄ 2 g/l, KH₂PO₄ 2 g/l, MgSO₄·7H₂O 0.5 g/l, (NH₄)SO₄ 0.5 g/l. The growing temperature is 37° C.±1° C. and the pH is maintained at 6.8±0.1 throughout the process by sterile addition of NH₄OH 25% or H₃PO₄ 35% automatically, stirring is 150 rpm. At the end of the fermentation, after approximately 20-24 h, the temperature is lowered to 4-8° C. to stop the cellular metabolism and harvest the broth.

The growth of the inoculum of Bifidobacterium and Lactobacillus is done in De Man, Rogosa, Sharpe Broth (MRS Broth) at 37° C. for 24 h. The fermentation is carried out at 37° C.±1° C., Ph 6.2±0.1 and 6.4±0.1, respectively, with the sterile addition of NH₄OH 25% or H₃PO₄ 35% automatically, and 50 rpm (the minimum value that guarantees the correct homogenization of the components of the medium). The culture medium is composed of Glucose H₂O 20 g/l, Yeast extract 5 g/l, K₂HPO₄ 2 g/l, Casein peptone 10 g/l, Milk powder 8 g/l, Sodium acetate 5 g/l, Diammonium citrate 2 g/l, Mn SO₄—H₂O 0.05 g/l, MgSO4 7H₂O 0.2 g/l, Tween 80 1 g/l. At the beginning, nitrogen is bubbled until the concentration of dissolved oxygen is at 0%, repeating the operation whenever necessary to maintain the O₂ at that level throughout the process. At the end of fermentation (20-24 h approx), it is lowered to 4-8° C. to stop cell metabolism and harvest the broth.

The inoculum of S. cerevisiae is grown in YPD for 24 h. In the fermenter the temperature is maintained at 30° C. and Ph 5 at 150 rpm and 1.5vvm. The fermentation is stopped in the fermenter itself, lowering the temperature to a value between 4-8° C.

The recovery of the biomass obtained based on the fermentation of each of the microorganisms is carried out by centrifugation or microfiltration, obtaining wet recovered biomasses, concentrating them as many times as necessary to reach between 1.00E+09 to 1.00E+11 CFU/ml in the concentrated biomasses of each microorganism.

Subsequently, the rupture or lysis of the cells of each of the wet recovered biomasses is carried out with a heat treatment and sonication, obtaining respective bacterial lysates that are subsequently dried separately in a lyophilizer or atomizer. The dry bacterial lysates are mixed in a mixer, to obtain the modulating composition.

Each batch of viable cell culture undergoes an autoclave sterilization cycle at 121° C. for 20 minutes. Subsequently, they are subjected to sonication for 20 min, at an output power of 500 W with an amplitude of 40% and 10 seconds of pause obtaining a mass of lysed cells of the probiotic (Qsonica, Q500). The final solution is freeze-dried and ground to obtain a lysate of the powdery probiotic microorganism. The powder is kept in a cool environment away from heat.

For the analysis of the lysates 0.5 gr of powder stock is taken from each batch and dissolved in 10 ml of distilled water. From this suspension a 1:8 dilution is prepared (100 ul sample and 700 ul water) and centrifuged for 5 minutes at 5000 rpm. The supernatant is collected, and 1.5 ul of sample is placed in a cuvette to measure absorbance at 260 nm, 230 nm and 280 nm in a Nanodrop Tecan Spark 10M, using distilled water as a target. The measure is at least duplicated and usually repeated more than four times.

The peak of maximum absorption for the quantification of nucleic acids, DNA, occurs at the wavelength of 260 nm, therefore, at that wavelength, the absorption will be proportional to the concentration of DNA. (ng/ul DNA: A260 nm×dilution factor×50 (conversion factor)

The amount of DNA present in each of the lysates is as follows:

TABLE 2
DNA concentrations of each of the dry lysates of bacterial strains
that were prepared in accordance with the procedure.

| Probiotic | DNA mg/g | SD |
|---|---|---|
| Lactobacillus casei | 13.68 | 1.89 |
| Lactobacillus acidophilus | 5.44 | 0.85 |
| Lactobacillus reuteri | 5.96 | 0.35 |
| Lactobacillus rhamnosus | 14.72 | 1.03 |
| Bifidobacterium lactis | 3.05 | 0.50 |
| Lactobacillus bulgaricus | 7.34 | 1.05 |
| Sreptococcus thermophilus | 10.71 | 1.65 |
| Bifidobacterium breve | 0.37 | |
| Bacillus subtilis | 6.68 | 0.99 |
| Bacillus licheniformis | 5.75 | 0.87 |
| Bacillus coagulans | 12.50 | |
| Bacillus mesentericus | 10.19 | 0.87 |
| Saccharomyces cereviseae | 12.01 | 1.93 |

A sample of the composition object of the invention was prepared with the following dry bacterial lysates in the following proportions:

TABLE 3
Sample composition 1 of the composition object of
the invention. The percentage by weight of each
of the genera described in this document is shown.

| | Sample 1 |
|---|---|
| Genera of dry bacterial lysates | (wt %) |
| Genus Bacillus | 16 |
| Genus Lactobacillus | 21 |
| Genus Streptococcus | 3 |
| Genus Saccharomyces | 52 |
| Genus Bifidobacterium | 8 |

### Example 2: MTT Assay and Determination of Telomerase Activity by QTRAP

### 2.1 MTT Assay

Through the present study, toxicity was determined by the MTT assay and the evaluation of the effect of a probiotic extract of Igen Biolab, the composition of which is indicated in Table 4, on telomerase activity by Q-TRAP in cultures of primary human fibroblasts.

TABLE 4
Composition of 1 sample of the composition object of the invention.
The percentage by weight of each of the bacterial species comprising
the dry bacterial lysates described in this document is shown. The
table also indicates the number of colony forming units.

| | % |
|---|---|
| Species | weight |
| Bacillus coagulans | 2 |
| Bacillus licheniformis | 6 |
| Bacillus mesentericus | 7 |
| Bacillus subtilis | 1 |
| Bifidobacterium breve | 6 |
| Bifidobacterium lactis | 2 |
| Lactobacillus acidophilus | 7 |
| Lactobacillus bulgaricus | 7 |
| Lactobacillus casei | 3 |
| Lactobacillus reuteri | 3 |
| Lactobacillus rhamnosus | 1 |
| Saccharomyces cereviseae | 52 |
| Streptococcus thermophilus | 3 |

### 2.1.1 Description of the Technique

The MTT toxicity assay is a colorimetric test to measure the metabolic activity of cells by serving as a substrate for cellular enzymes that reduce the orange tetrazolium dye, MTT (3-(4,5-Dimethylthiazol-2-yl)-2,5-diphenyltetrazolium bromide) to its insoluble formazan, giving a purple color. The conversion rate is directly proportional to the mitochondrial metabolic activity of cells via NAD (P) H-dependent cellular oxidoreductase enzymes and serves as a surrogate marker of cell viability.

### 2.1.2 Cellular and Culture Conditions

The stock of primary adult human fibroblast cultures (3 passages) was established under standard culture conditions. The cells were seeded at 3×10³ cells/^{cm2} in a fibroblast medium (Innoprot kit).

Fibroblast Medium (FM) is a complete medium designed for the optimal growth of human fibroblasts in vitro. It is a sterile liquid medium containing essential and non-essential amino acids, vitamins, organic and inorganic compounds, hormones, growth factors, trace minerals and a low concentration of fetal bovine serum (2%). The medium is HEPES and bicarbonate buffer system with a pH of 7.4 after being balanced in an incubator with an atmosphere of 5% CO₂/95%. The medium is formulated (quantitatively and qualitatively) to provide a defined and optimally balanced nutritional environment that selectively promotes the proliferation and growth of normal human fibroblasts in vitro.

Previously expanded cells were seeded into 96-well plates (Nunc) at 0.5×10⁴ cells/plate and 0.35×10⁴ cells/plate during a 72-hour and one-week treatment, respectively. This concentration shows the best window for the MTT assay, with better sensitivity and low variability.

The compound was supplied in dry powder form and remained in optimal condition until use. An amount of 18 mg of the compound (powder) was dissolved in 18 ml (1 mg/ml) of 20% DMSO at room temperature. Eight-point curves were prepared, dilutions 1/2 (highest concentration 1 mg/ml). The final DMSO concentration was 0.5% (this concentration does not affect cells).

Twenty-four hours after the seeding process, the cells were washed once with PBS and treated with the compounds in their respective cell culture media (fibroblast media kit). Each condition was analyzed in triplicate with and without H₂O₂ 10 μM. For positive and negative controls, methyl methane sulfonate (MMS) 8 mM and 100% DMSO were used, respectively.

After the addition of the compound, the plates were incubated for 72 hours and a week, while the medium with the compound was changed every other day. After the treatment period, the cells were washed twice with PBS and the medium was replaced with MTT reagent at 0.5 mg/ml in DMEM without phenol red. The plates were gently shaken and incubated for 4 hours. After incubation, the medium was removed and replaced by DMSO. The plates were gently shaken to solubilize the formazan crystals.

Absorbance was measured using the Envision multi-plate reader at a wavelength of 570 nm.

### 2.1.3 Results

Toxicity Test (MTT) in fibroblasts at 72 hours and one week.

Eight serial dilutions (50 μg/ml-16.7 μg/ml-5.6 μg/ml-1.9 μg/ml-0.6 μg/ml-0.2 μg/ml-0.1 μg/ml-0.02 μg/ml) were prepared to evaluate the compound.

They were prepared tripled by concentration and tested under standard and oxidative stress conditions to evaluate the compound.

MTT results showing cell death values above 20% are considered to have a significant toxic effect.

TABLE 5
MTT assay results expressed as the percentage of cell death
after treatment, after 72 hours under standard conditions.

| | Cell | Cell | Cell | | |
|---|---|---|---|---|---|
| Concentration | death #1 | death #2 | death #3 | | |
| (μg/ml) | (%) | (%) | (%) | Mean | SD |
| 50.0 | −19.41 | −30.17 | −28.02 | −25.86 | 5.69 |
| 16.7 | 2.63 | 9.40 | 6.42 | 6.15 | 3.39 |
| 5.6 | 1.50 | −6.08 | −1.06 | −1.88 | 3.86 |
| 1.9 | 14.01 | 12.27 | 12.37 | 12.88 | 0.98 |
| 0.6 | −15.72 | −11.10 | −4.54 | −10.45 | 5.61 |
| 0.2 | 6.22 | 12.37 | 17.70 | 12.09 | 5.74 |
| 0.1 | −9.05 | −7.72 | −2.29 | −6.35 | 3.58 |
| 0.02 | 0.48 | 2.32 | 8.37 | 3.72 | 4.13 |

TABLE 6
MTT assay results expressed as the percentage of cell death
after treatment, after 72 hours on oxidative stress.

| | Cell | Cell | Cell | | |
|---|---|---|---|---|---|
| Concentration | death #1 | death #2 | death #3 | | |
| (μg/ml) | (%) | (%) | (%) | Mean | SD |
| 50.0 | −21.76 | −19.61 | −17.15 | −19.51 | 2.31 |
| 16.7 | 12.68 | 10.83 | 12.57 | 12.03 | 1.04 |
| 5.6 | −4.75 | −8.54 | −5.77 | −6.35 | 1.96 |
| 1.9 | 6.12 | 11.24 | 12.06 | 9.81 | 3.22 |
| 0.6 | −9.05 | −11.92 | −18.38 | −13.12 | 4.78 |
| 0.2 | −0.85 | 3.86 | −4.34 | −0.44 | 4.12 |
| 0.1 | −10.28 | −18.38 | −14.69 | −14.45 | 4.05 |
| 0.02 | −3.31 | −0.85 | −2.49 | −2.22 | 1.25 |

TABLE 7
MTT assay results expressed as the percentage of cell death
after treatment, after one week under standard conditions.

| | Cell | Cell | Cell | | |
|---|---|---|---|---|---|
| Concentration | death #1 | death #2 | death #3 | | |
| (μg/ml) | (%) | (%) | (%) | Mean | SD |
| 50.0 | −6.14 | 6.19 | 13.04 | 4.36 | 9.72 |
| 16.7 | 6.93 | 1.45 | 11.25 | 6.54 | 4.91 |
| 5.6 | 7.24 | −8.88 | 6.19 | 1.52 | 9.02 |
| 1.9 | 9.14 | 15.36 | 12.72 | 12.41 | 3.12 |
| 0.6 | 0.08 | 6.19 | 7.67 | 4.65 | 4.02 |
| 0.2 | 5.87 | 13.25 | 13.15 | 10.76 | 4.23 |
| 0.1 | 1.98 | 9.56 | 15.04 | 8.86 | 6.56 |
| 0.02 | 3.87 | 5.66 | 18.41 | 9.32 | 7.93 |

TABLE 8
MTT assay results expressed as the percentage of cell death
after treatment, after one week on oxidative stress.

| | Cell | Cell | Cell | | |
|---|---|---|---|---|---|
| Concentration | death #1 | death #2 | death #3 | | |
| (μg/ml) | (%) | (%) | (%) | Mean | SD |
| 50.0 | 14.94 | 9.04 | 15.99 | 13.32 | 3.75 |
| 16.7 | 7.24 | −7.30 | 11.04 | 3.66 | 9.68 |
| 5.6 | 5.45 | 13.78 | −8.77 | 3.49 | 11.40 |
| 1.9 | 10.41 | −2.45 | 5.45 | 4.47 | 6.48 |
| 0.6 | 5.56 | 16.94 | 3.03 | 8.51 | 7.41 |
| 0.2 | 13.67 | 5.03 | 16.52 | 11.74 | 5.98 |
| 0.1 | 12.30 | 1.34 | 20.42 | 11.35 | 9.57 |
| 0.02 | 15.78 | 11.04 | 7.56 | 11.46 | 4.13 |

### 2.1.4 Results and Observations

No precipitation of the compound was observed at the tested concentrations. According to the results, the concentrations of the composition of the invention chosen for the Q-TRAP and TAT assays were 50, 16.7 and 5.6 μg/ml.

### 2.2. Determination of Telomerase Activity by Q-TRAP

This assay determines telomerase activity by Q-TRAP in adult human fibroblast primary cultures after 6, 24, 48 and 72 hours of treatment at the above concentrations.

### 2.2.1 Description of the Technique

Q-TRAP can measure relative telomerase activity using the Telomere Repeated Amplification Protocol (TRAP), modified for quantitative real-time PCR analysis (Q-TRAP). This method has the advantages of greater sensitivity, speed and a high-performance format compared to the normal TRAP assay. The activity of the enzyme telomerase in lysates of whole cells of cell cultures is evaluated.

The general mechanism of the Q-TRAP technique consists of the lysis of cell granules for the extraction of proteins, which are then quantified and stored under specific conditions to prevent their degradation. The protein obtained in the process was used within 24 hours, the samples were stored at 4° C. Telomerase protein extracts are incubated with a specific oligonucleotide substrate to allow enzymatic addition of telomeric DNA repetitions by endogenous telomerase.

After the enzymatic reaction, telomerase extension products are amplified and quantified by real-time qPCR. By qPCR, a positive reaction is detected by the accumulation of the fluorescent signal. The Ct (cycle threshold) is defined as the number of cycles required for the fluorescence to cross the threshold (i.e. exceed background levels). The telomerase-positive standard dilution series is plotted against telomere protein concentration (r2>0.9) as a standard curve of Ct values.

To ensure that the data are both reproducible and quantitative, the test is performed in triplicate. The mean and standard deviation (SD) of each triplicate are calculated, which include both positive (standard lymphoid cell line curve) and negative (heat-inactivated) controls.

The data is reported as RTA (relative telomerase activity) and the overall workflow scheme can be seen below.

### 2.2.2 Results

### Quality Control Parameters

Before seeding and performing the Q-TRAP protocol, the samples were evaluated for:
Protein concentration: protein quantification is performed on each sample using the Biorad protein assay. A minimum protein concentration of 0.3 μg/μl is required to proceed with sample analysis to ensure consistent results.
Regression curve: internal controls are included, and a regression analysis is performed for each cycle/plate. It is repeated in the plates in which the regression curves have an R2 less than 0.9
Replicas: Samples with less than 2 valid replicas are discarded.
Amplification cycle: signals obtained after cycle #35 are considered nonspecific amplification.

TABLE 9
Abbreviations used throughout this report

| Treatment | Abbreviation |
|---|---|
| Untreated control | Ctrl |
| Treatment with IG at 50 mg/ml | IG_1 |
| Treatment with 16.7 ug/ml | IG_2 |
| Treatment with 5.6/ml | IG_3 |

### Protein Concentration.

TABLE 10
Protein concentration results for samples analyzed with Q-
TRAP. Each column shows the concentration of each triplicate.

| Treatment | Time (h) | Protein (μg/ul) |
|---|---|---|
| Control- Time 0 Replica 1 | 0 | 0.64 |
| Control- Time 0 Replica 2 | 0 | 0.66 |
| Control- Time 0 Replica 3 | 0 | 0.74 |
| Control- 6 hours Replica 1 | 6 | 0.58 |
| Control- 6 hours Replica 2 | 6 | 0.80 |
| Control- 6 hours Replica 3 | 6 | 0.81 |
| IG_1 -6 hours - Replica 1 | 6 | 0.71 |
| IG_1 -6 hours - Replica 2 | 6 | 0.81 |
| IG_1 -6 hours - Replica 3 | 6 | 1.12 |
| IG_2 -6 hours - Replica 1 | 6 | 0.82 |
| IG_2 -6 hours - Replica 2 | 6 | 0.65 |
| IG_2 -6 hours - Replica 3 | 6 | 1.12 |
| IG_3 -6 hours - Replica 1 | 6 | 0.80 |
| IG_3 -6 hours - Replica 2 | 6 | 0.66 |
| IG_3 -6 hours - Replica 3 | 6 | 1.32 |
| Control- 12 hours -Replica 1 | 12 | 0.73 |
| Control- 12 hours -Replica 2 | 12 | 0.88 |
| Control- 12 hours -Replica 3 | 12 | 0.79 |
| IG_1 -12 hours - Replica 1 | 12 | 0.72 |
| IG_1 -12 hours - Replica 2 | 12 | 1.04 |
| IG_1 -12 hours - Replica 3 | 12 | 0.99 |
| IG_2 -12 hours - Replica 1 | 12 | 0.89 |
| IG_2 -12 hours - Replica 2 | 12 | 0.73 |
| IG_2 -12 hours - Replica 3 | 12 | 0.94 |
| IG_3 -12 hours - Replica 1 | 12 | 0.95 |
| IG_3 -12 hours - Replica 2 | 12 | 0.73 |
| IG_3 -12 hours - Replica 3 | 12 | 0.99 |
| Control- 24 hours - Replica 1 | 24 | 0.81 |
| Control- 24 hours - Replica 2 | 24 | 0.81 |
| Control- 24 hours - Replica 3 | 24 | 0.95 |
| IG_1 -24 hours - Replica 1 | 24 | 0.84 |
| IG_1 -24 hours - Replica 2 | 24 | 0.84 |
| IG_1 -24 hours - Replica 3 | 24 | 0.86 |
| IG_2 -24 hours - Replica 1 | 24 | 0.78 |
| IG_2 -24 hours - Replica 2 | 24 | 0.91 |

### 2.2.3 Results of Telomerase Activity

Only those samples with sufficient protein (>0.3 mg/ml) were analyzed. Standard curve results: generated by graphically representing the threshold cycles (Ct values) of the HeLa cell line standards versus the registry.

The measurements were made in triplicate to calculate the coefficients of variation and the mean of the amplification signals.

The following table summarizes the average data in triplicate. Mean and SD after normalization versus Hela control were reported as relative telomerase activity (RTA) for each sample.

TABLE 11
RTA values for each sample analyzed.

| Trt. | 0 hours | | 6 hours | | 12 hours | | 24 hours | | 72 hours | |
|---|---|---|---|---|---|---|---|---|---|---|
| (μg/ml) | RTA % | SD | RTA % | SD | RTA % | SD | RTA % | SD | RTA % | SD |
| Control | 20.35 | 0.39 | 30.46 | 5.04 | 12.82 | 0.69 | 21.1 | 0.86 | 14.93 | 1.27 |
| IG_1 | | | 40.10 | 1.42 | 15.81 | 4.49 | 22.6 | 2.13 | 16.93 | 2.12 |
| IG_2 | | | 27.67 | 5.12 | 16.13 | 6.10 | 122.3 | 3.86 | 20.52 | 0.48 |
| IG_3 | | | 18.97 | 0.05 | 13.25 | 0.98 | 20.8 | 1.04 | 17.10 | 1.03 |

### 2.2.4 Statistical Analysis

Q-TRAP analyses for primary fibroblasts of human adults at different times were compared. Data were pooled by condition and timing of treatment. Statistical analysis was applied for pooled data using T-student compared to the control condition.

TABLE 12
The T-Student analysis indicates whether there are significant
differences between the QTRAP results compared to the control group.
T-Student Analysis - Relative telomerase activity (RTA)

| | RTA (p Value) | Significance |
|---|---|---|
| 6 hours | | |
| Control vs. IG_1 | 1.21E−01 | NO |
| Control vs. IG_2 | 5.90E−01 | NO |
| Control vs. IG_3 | 8.42E−02 | NO |
| 12 hours | | |
| Control vs. IG_1 | 3.05E−01 | NO |
| Control vs. IG_2 | 3.85E−01 | NO |
| Control vs. IG_3 | 5.67E−01 | NO |
| 24 hours | | |
| Control vs. IG_1 | 3.40E−01 | NO |
| Control vs. IG_2 | 6.38E−01 | NO |
| Control vs. IG_3 | 6.85E−01 | NO |
| 72 hours | | |
| Control vs. IG_1 | 2.34E−01 | NO |
| Control vs. IG_2 | 1.07E−02 | YES (*) |
| Control vs. IG_3 | 1.41E−01 | NO |

Significant differences are indicated in the “Significance” column. Lowest to highest: No: not significant; Yes (*): p < 0.05; Yes (**): p < 0.01; Yes (***): p < 0.001; Yes (****): p < 0.0001.

### 2.2.5 Observations and Conclusions

### Quality Control Results

Protein extraction yields from all samples generated an adequate amount of protein (>0.3 μg/μl) to perform the Q-TRAP assay.
The coefficient of determination of the regression curve (HeLa cells) was greater than 0.9 (R2=0.99).

### 2.2.5. Results of Telomerase Activity

Group IG_1 has a higher RTA than the control group for all the times. Although the differences are not statistically significant, the effect maintained on activation for all points shows a consistent positive effect for the IG_1 group.
The IG_2 group presents a telomerase activation for the times 12, 24 and 72 hours.
The IG_3 group presents higher RTA compared to the untreated control for times 12 and 72.

### Example 3: Proliferation Analysis in Cell Culture and Telomere Length Measurements by TAT®

### 3.1. Proliferation Analysis in Cell Culture

### 3.1.1. Description of the Technique

Primary cultures of adult human fibroblast cells were established. The cells are seeded at 5×10³ cells/cm², in a fibroblast medium kit (Innoprot). Fibroblast Medium (FM) is a complete medium designed for optimal growth of normal human fibroblasts in vitro. It is a sterile liquid medium containing essential and non-essential amino acids, vitamins, organic and inorganic compounds, hormones, growth factors, trace minerals and a low concentration of fetal bovine serum (2%). The medium contains HEPES and buffered bicarbonate and has a pH of 7.4 when balanced in an incubator with an atmosphere of 5% CO₂/95% of air. The medium is formulated (quantitatively and qualitatively) to provide a defined and balanced nutritional environment that selectively promotes the proliferation and growth of normal human fibroblasts in vitro.

The media are renewed every 2-3 days and the cells are passed in subconfluence (70-80%) every 7 days. Compounds or treatment are added to cells in culture. Cell growth is monitored for each condition by counting the number of cells at each step using a Countess™ (Invitrogen) cell counter. Population doubling (PD) was calculated with the formula PD=3.322 (Log (Cf)−Log (Ci))+x (Cf: Final concentration; Ci, initial concentration; X: PD last passage). A PD is equivalent to one round of cell replication.

### 3.1.2. Materials and Methods

### Treatments

The different treatments were evaluated in the proliferation analysis:

TABLE 13
Abbreviations used in the report.

| Treatment | Abbreviation |
|---|---|
| Standard growing conditions (Ctrl) | Ctrl |
| Standard culture conditions + IG at | IG_1 |
| 50 ug/ml | |
| Standard culture conditions + IG at | IG_2 |
| 16.6 ug/ml | |
| Standard culture conditions + IG at | IG_3 |
| 5.6 ug/ml | |
| Oxidative stress culture conditions = | H₂O₂ |
| Standard conditions + 10 μM H2O2 | |
| Culture conditions oxidative stress + | IG_1 + H₂O₂ |
| IG at 50 ug/ml | |
| Culture conditions oxidative stress + | IG_2 + H₂O₂ |
| IG at 16.6 ug/ml | |
| Culture conditions oxidative stress + | IG_3 + H₂O₂ |
| IG at 5.6 ug/ml | |

The cells were expanded for eight weeks under standard and oxidative stress conditions (H₂O₂).

### 3.1.3. Results

TABLE 14
Cumulative Population Doubling per passage (mean
of three replicates) under standard conditions.
Cumulative Population Doubling

| Week | 0 | 1 | 2 | 3 | 4 | 5 | 6 | 7 | 8 |
|---|---|---|---|---|---|---|---|---|---|
| Control | 0 | 2.53 | 5.45 | 8.36 | 11.20 | 14.04 | 16.33 | 18.58 | 20.89 |
| IG_1 | 0 | 2.62 | 6.05 | 9.15 | 12.13 | 15.09 | 17.50 | 19.34 | 21.54 |
| IG_2 | 0 | 2.83 | 6.20 | 9.41 | 12.47 | 15.09 | 17.61 | 19.82 | 22.28 |
| IG_3 | 0 | 2.94 | 6.15 | 9.28 | 12.19 | 14.79 | 17.17 | 19.42 | 21.84 |

TABLE 15
Cumulative Population Doubling per passage (mean
of three replicates) under conditions of oxidative stress.
Cumulative Population Doubling

| Week | 0 | 1 | 2 | 3 | 4 | 5 | 6 | 7 | 8 |
|---|---|---|---|---|---|---|---|---|---|
| H2O2 | 0 | 2.80 | 5.98 | 8.89 | 11.90 | 14.72 | 17.19 | 19.36 | 22.14 |
| IG_1 + H2O2 | 0 | 3.10 | 6.76 | 10.15 | 12.99 | 16.18 | 18.84 | 21.08 | 23.22 |
| IG_2 + H2O2 | 0 | 2.99 | 6.55 | 9.90 | 12.97 | 16.04 | 18.91 | 21.44 | 24.09 |
| IG_3 + H2O2 | 0 | 3.04 | 6.28 | 9.60 | 12.72 | 15.58 | 18.11 | 20.49 | 22.85 |

### 3.1.4. Observations and Conclusions

After 8 weeks of treatment at determined strengths:

### Under Standard Conditions:

No significant differences were observed in the proliferation of cells treated with IG_1, IG_2 and IG_3 at different concentrations during the 8 weeks of expansion.

### Under Conditions of Oxidative Stress:

No significant differences were observed in the proliferation of cells treated with IG_1, IG_2 and IG_3 at different concentrations during the 8 weeks of expansion

### 3.2. Telomere Length Measurements Using TAT®

### 3.2.1 Description of the Technique

To measure the median telomere length of any cell line, Life Length uses a high-throughput (HT) Q-FISH technique. This method is based on an in-situ hybridization method with modified quantitative fluorescence for interphase cells. In summary, telomeres are hybridized with a fluorescent peptide nucleic acid (PNA) probe that recognizes three telomere repetitions (sequence: Alexa488-OO-CCCTAACCCTAACCCTAAA, Panagene). Images of nuclei and telomeres are captured by a high-content display system (see below). The fluorescent signal strength of telomeric PNA probes that hybridize with each telomere is proportional to the length of that telomere. Fluorescence intensities are translated into base pairs through a standard regression curve that is generated using control cell lines with known telomere length.

Sample preparation and HT Q-FISH: On the day of processing, samples and control cell lines frozen in liquid nitrogen are thawed at 37° C. and cell counts and viability are determined. Aliquots with a viability of less than 60% are considered below our quality control standards and will not be further analyzed. Cells are seeded into 384-well plates at a density of 15,000 cells per well with 5 replicates of each sample and 8 replicates of each control cell line. Two identical independent plates are prepared for each set of samples. The cells are fixed with methanol/acetic acid (3/1, vol/vol). Once the cells have fixed themselves in the plate, they are treated with pepsin to digest the cytoplasm and the nuclei are processed for in-situ hybridization with the PNA probe. After several washing steps and incubation with standard DAPI for DNA staining, the wells are filled with mounting medium and the plate is stored overnight at 4° C.

HT Microscopy: Quantitative image acquisition and analysis is performed on an Opera Phenix high-content screening system (Perkin Elmer), using Columbus software, version 2.9 (Perkin Elmer). Images are captured using a 40×0.95 NA water immersion lens. UV wavelengths excitation and 488 nm are used to detect the DAPI and A488 signals respectively. With constant exposure settings, 15 independent images are captured in different positions for each well. The images of the nuclei are then used to define the region of interest of each cell, measuring the fluorescence intensity of the telomeres of the A488 image in all of them. The intensity results for each focus are exported to the Columbus 2.4 (Perkin Elmer) software. Telomere length distribution and median telomere length are calculated with Life Length's proprietary software. The statistical analysis of the data was performed using the T-student test.

### 3.2.2 Technology Validation

TAT technology has been validated for the following parameters:

Accuracy: Matching TAT fluorescence intensity values to telomere length measurements is achieved by performing TRF (terminal restriction fragmentation) on six human lymphocyte cell lines (calibration/method comparison). The same set of samples is analyzed by both TAT and the TRF (Definition of Systemic Error TAT) reference method.

VALIDATION DATA shows a correlation of 0.99.

An analysis of the values of the median telomere length is performed in a sample of human lymphocytes in different series, days and positions of the plate to define the parameters of random error TAT (standard deviation, variance).

VALIDATION DATA indicates that TAT has a standard deviation of 454 base pairs.

Limit of detection and specificity: There are definitions of image analysis algorithms and protocol configurations that define the lowest significant intensities and prevent interference from non-specific fluorescence signals.

VALIDATION DATA defines the detection limit at 800 base pairs and demonstrates very high specificity.

Reportable range of the median: an analysis of the median telomere length of 6 cell lines is performed that covers the reportable range and defines its lower and upper limits.

VALIDATION DATA sets the lower level at 4,700 base pairs and the upper level at 14,400 base pairs.

Reference range: The analysis of median telomere length has been evaluated in hundreds of human samples to define the reference range of TAT and its percentiles (5th, 10th, 25th, 50th, 75th and 95th) for the different ages.

The VALIDATION DATA established population curves—normal population curve from 18 to 85 years, to extrapolate and generate reports.

### 3.2.3 Results

### Quality Control Parameters

During the TAT protocol, samples were analyzed for:
Cell count: An automated cell counter is used to determine the total number of cells in the vials.
Cell viability by Tripan-Blue's exclusive method
Regression curve: Internal controls are included, and a regression analysis is performed for each sample/plate. It is repeated in the plates where the regression curve has an R2 less than 0.92.

Replicated: Once the sowing and testing of TAT is carried out:

Samples must have a CV below 10%.

Samples with less than 3 replicated at the end of the analysis are discarded.

The number of spots analyzed per sample must be greater than 10,000.

### Number of Cells and Viability.

TABLE 16
Concentration and viability of the samples after thawing
the triplicates under standard conditions.

| | | Concentration | |
|---|---|---|---|
| | | Cellular | |
| | Treatment and Time | (cell/ml) | Viability % |
| | Control - Week 0 - Replicate 1 | 0.53 | 95 |
| | Control - Week 0 - Replicate 2 | 0.53 | 99 |
| | Control - Week 0 - Replicate 3 | 0.72 | 94 |
| Week | Control - Week 2 - Replicate 1 | 0.92 | 85 |
| 2 | Control - Week 2 - Replicate 2 | 0.98 | 97 |
| | Control - Week 2 - Replicate 3 | 0.85 | 95 |
| | IG_1 - Week 2 - Replicate 1 | 1.00 | 97 |
| | IG_1 - Week 2 - Replicate 2 | 1.10 | 97 |
| | IG_1 - Week 2 - Replicate 3 | 1.10 | 96 |
| | IG_2 - Week 2 - Replicate 1 | 1.20 | 95 |
| | IG_2 - Week 2 - Replicate 2 | 0.98 | 94 |
| | IG_2 - Week 2 - Replicate 3 | 0.80 | 96 |
| | IG_3 - Week 2 - Replicate 1 | 0.82 | 97 |
| | IG_3 - Week 2 - Replicate 2 | 1.10 | 98 |
| | IG_3 - Week 2 - Replicate 3 | 0.83 | 87 |
| Week | Control - Week 4 - Replicate 1 | 0.93 | 97 |
| 4 | Control - Week 4 - Replicate 2 | 0.95 | 92 |
| | Control - Week 4 - Replicate 3 | 0.80 | 95 |
| | IG_1 - Week 4 - Replicate 1 | 0.99 | 88 |
| | IG_1 - Week 4 - Replicate 2 | 0.73 | 96 |
| | IG_1 - Week 4 - Replicate 3 | 0.77 | 97 |
| | IG_2 - Week 4 - Replicate 1 | 0.98 | 91 |
| | IG_2 - Week 4 - Replicate 2 | 0.75 | 97 |
| | IG_2 - Week 4 - Replicate 3 | 0.77 | 97 |
| | IG_3 - Week 4 - Replicate 1 | 1.20 | 90 |
| | IG_3 - Week 4 - Replicate 2 | 0.86 | 95 |
| | IG_3 - Week 4 - Replicate 3 | 0.80 | 99 |
| Week | Control - Week 6 - Replicate 1 | 0.71 | 72 |
| 6 | Control - Week 6 - Replicate 2 | 0.57 | 99 |
| | Control - Week 6 - Replicate 3 | 0.31 | 98 |
| | IG_1 - Week 6 - Replicate 1 | 0.69 | 99 |
| | IG_1 - Week 6 - Replicate 2 | 0.70 | 94 |
| | IG_1 - Week 6 - Replicate 3 | 0.55 | 97 |
| | IG_2 - Week 6 - Replicate 1 | 0.68 | 100 |
| | IG_2 - Week 6 - Replicate 2 | 0.72 | 100 |
| | IG_2 - Week 6 - Replicate 3 | 0.81 | 96 |
| | IG_3 - Week 6 - Replicate 1 | 0.52 | 99 |
| | IG_3 - Week 6 - Replicate 2 | 0.76 | 91 |
| | IG_3 - Week 6 - Replicate 3 | 0.86 | 94 |
| Week | Control - Week 8 - Replicate 1 | 0.40 | 94 |
| 8 | Control - Week 8 - Replicate 2 | 0.44 | 96 |
| | Control - Week 8 - Replicate 3 | 0.43 | 99 |
| | IG_1 - Week 8 - Replicate 1 | 0.37 | 91 |
| | IG_1 - Week 8 - Replicate 2 | 0.44 | 89 |
| | IG_1 - Week 8 - Replicate 3 | 0.33 | 97 |
| | IG_2 - Week 8 - Replicate 1 | 0.75 | 80 |
| | IG_2 - Week 8 - Replicate 2 | 0.47 | 98 |
| | IG_2 - Week 8 - Replicate 3 | 0.54 | 80 |
| | IG_3 - Week 8 - Replicate 1 | 0.46 | 100 |
| | IG_3 - Week 8 - Replicate 2 | 0.34 | 96 |
| | IG_3 - Week 8 - Replicate 3 | 0.37 | 87 |

TABLE 17
Concentration and viability of the samples after
thawing the triplicates in oxidative stress.

| | | Cell | |
|---|---|---|---|
| | | Concentration | |
| | Treatment and Time Point | (cell/ml) | Viability % |
| Week | Control - Week 2 - Replicate 1 | 1.20 | 94 |
| 2 | Control - Week 2 - Replicate 2 | 0.54 | 97 |
| | Control - Week 2 - Replicate 3 | 0.67 | 99 |
| | IG_1 - Week 2 - Replicate 1 | 1.90 | 94 |
| | IG_1 - Week 2 - Replicate 2 | 1.20 | 99 |
| | IG_1 - Week 2 - Replicate 3 | 0.63 | 97 |
| | IG_2 - Week 2 - Replicate 1 | 1.20 | 97 |
| | IG_2 - Week 2 - Replicate 2 | 1.10 | 94 |
| | IG_2 - Week 2 - Replicate 3 | 1.10 | 99 |
| | IG_3 - Week 2 - Replicate 1 | 1.10 | 97 |
| | IG_3 - Week 2 - Replicate 2 | 0.96 | 98 |
| | IG_3 - Week 2 - Replicate 3 | 0.57 | 95 |
| Week | Control - Week 4 - Replicate 1 | 1.10 | 93 |
| 4 | Control - Week 4 - Replicate 2 | 1.00 | 94 |
| | Control - Week 4 - Replicate 3 | 0.73 | 94 |
| | IG_1 - Week 4 - Replicate 1 | 0.85 | 99 |
| | IG_1 - Week 4 - Replicate 2 | 0.81 | 93 |
| | IG_1 - Week 4 - Replicate 3 | 0.82 | 98 |
| | IG_2 - Week 4 - Replicate 1 | 0.95 | 96 |
| | IG_2 - Week 4 - Replicate 2 | 1.10 | 95 |
| | IG_2 - Week 4 - Replicate 3 | 1.70 | 99 |
| | IG_3 - Week 4 - Replicate 1 | 0.96 | 99 |
| | IG_3 - Week 4 - Replicate 2 | 0.74 | 94 |
| | IG_3 - Week 4 - Replicate 3 | 2.60 | 99 |
| Week | Control - Week 6 - Replicate 1 | 0.75 | 97 |
| 6 | Control - Week 6 - Replicate 2 | 0.64 | 85 |
| | Control - Week 6 - Replicate 3 | 0.42 | 88 |
| | IG_1 - Week 6 - Replicate 1 | 0.51 | 100 |
| | IG_1 - Week 6 - Replicate 2 | 0.44 | 100 |
| | IG_1 - Week 6 - Replicate 3 | 0.55 | 94 |
| | IG_2 - Week 6 - Replicate 1 | 0.46 | 96 |
| | IG_2 - Week 6 - Replicate 2 | 0.51 | 99 |
| | IG_2 - Week 6 - Replicate 3 | 0.55 | 92 |
| | IG_3 - Week 6 - Replicate 1 | 0.43 | 100 |
| | IG_3 - Week 6 - Replicate 2 | 0.82 | 93 |
| | IG_3 - Week 6 - Replicate 3 | 0.62 | 99 |
| Week | Control - Week 8 - Replicate 1 | 0.63 | 98 |
| 8 | Control - Week 8 - Replicate 2 | 0.39 | 95 |
| | Control - Week 8 - Replicate 3 | 0.48 | 89 |
| | IG_1 - Week 8 - Replicate 1 | 0.34 | 84 |
| | IG_1 - Week 8 - Replicate 2 | 0.35 | 97 |
| | IG_1 - Week 8 - Replicate 3 | 0.37 | 85 |
| | IG_2 - Week 8 - Replicate 1 | 0.27 | 98 |
| | IG_2 - Week 8 - Replicate 2 | 0.34 | 88 |
| | IG_2 - Week 8 - Replicate 3 | 0.33 | 100 |
| | IG_3 - Week 8 - Replicate 1 | 0.38 | 91 |
| | IG_3 - Week 8 - Replicate 2 | 0.40 | 87 |
| | IG_3 - Week 8 - Replicate 3 | 0.18 | 100 |

### Telomere Length Results by TAT

The table below shows the median telomere length and median telomere length of the 20th percentile (both in base pairs, bp) for each sample, as well as the percentage of short telomeres. The latter is defined as the percentage of telomeres with a length less than 3 Kbp (<3 Kbp). All measurements were performed fivefold.

TABLE 18
Telomere length results of the TAT
analysis under standard conditions.

| | Median | 20th | Telomeres <3 | |
|---|---|---|---|---|
| Treatment and Time | (base | percentile | Kbp | CV |
| Points | pair) | (base pairs) | (%) | (%) |
| Control - Week 0 | 9545 | 6533 | 4.02 | 1.64 |
| Control - Week 2 | 9483 | 6367 | 4.73 | 2.65 |
| IG_1 - Week 2 | 9313 | 6218 | 4.58 | 0.40 |
| IG_2 - Week 2 | 9612 | 6559 | 3.69 | 0.46 |
| IG_3 - Week 2 | 9564 | 6474 | 4.27 | 1.61 |
| Control - Week 4 | 9355 | 6161 | 5.35 | 0.38 |
| IG_1 - Week 4 | 9089 | 5973 | 4.95 | 0.69 |
| IG_2 - Week 4 | 9251 | 6138 | 4.82 | 2.21 |
| IG_3 - Week 4 | 8799 | 5586 | 7.05 | 0.52 |
| Control - Week 6 | 8540 | 5412 | 7.03 | 3.58 |
| IG_1 - Week 6 | 8447 | 5263 | 7.62 | 0.35 |
| IG_2 - Week 6 | 8662 | 5502 | 6.83 | 1.21 |
| IG_3 - Week 6 | 8647 | 5473 | 7.12 | 0.24 |
| Control - Week 8 | 8308 | 5039 | 8.24 | 2.59 |
| IG_1 - Week 8 | 7072 | 3837 | 14.31 | 2.81 |
| IG_2 - Week 8 | 8308 | 5037 | 8.38 | 0.13 |
| IG_3 - Week 8 | 8312 | 5085 | 7.91 | 1.64 |

TABLE 19
Telomere length results of TAT analysis
under oxidative stress conditions

| | | 20th | Telo- | |
|---|---|---|---|---|
| | Median | percentile | meros <3 | |
| Treatment and Time | (base | (base | Kbp | CV |
| Points | pairs) | pairs) | (%) | (%) |
| 10 μM H2O2 - Week 2 | 9332 | 6236 | 4.48 | 2.26 |
| IG_1 + 10 μM H2O2 - Week 2 | 8978 | 5876 | 5.87 | 0.56 |
| IG_2 + 10 μM H2O2 - Week 2 | 8828 | 5644 | 6.87 | 2.60 |
| IG_3 + 10 μM H2O2 - Week 2 | 9178 | 6047 | 5.25 | 2.86 |
| 10 μM H2O2 - Week 4 | 8782 | 5554 | 7.41 | 3.33 |
| IG_1 + 10 μM H2O2 - Week 4 | 9044 | 5908 | 5.23 | 1.27 |
| IG_2 + 10 μM H2O2 - Week 4 | 8998 | 5853 | 5.62 | 1.01 |
| IG_3 + 10 μM H2O2 - Week 4 | 8931 | 5734 | 6.16 | 1.66 |
| 10 μM H2O2 - Week 6 | 8700 | 5509 | 7.02 | 0.95 |
| IG_1 + 10 μM H2O2 - Week 6 | 8470 | 5260 | 7.46 | 3.95 |
| IG_2 + 10 μM H2O2 - Week 6 | 8449 | 5202 | 7.72 | 2.32 |
| IG_3 + 10 μM H2O2 - Week 6 | 8722 | 5500 | 6.74 | 0.57 |
| 10 μM H2O2 - Week 8 | 8444 | 5184 | 7.77 | 3.66 |
| IG_1 + 10 μM H2O2 - Week 8 | 7957 | 4709 | 9.59 | 1.15 |
| IG_2 + 10 μM H2O2 - Week 8 | 7665 | 4429 | 10.81 | 2.86 |
| IG_3 + 10 μM H2O2 - Week 8 | 8194 | 4958 | 8.54 | 3.23 |

### 3.2.4 Analysis of Results

### Standard Growing Conditions

Because cell replication is one of the main causes of telomere shortening, measured telomeric lengths were normalized by population doubling for each condition and time.

TABLE 20
Telomere shortening ratio (Median telomere length
(initial − final)/Population Doubling) for
treatments and times defined under standard conditions.
Telomere shortening ratio - Standard conditions

| Treatment | Week 2 | Week 4 | Week 6 | Week 8 |
|---|---|---|---|---|
| Control | 36.0 | 14.4 | 52.3 | 73.4 |
| IG_1 | 38.4 | 37.6 | 65.9 | 93.9 |
| IG_2 | −11.0 | 33.2 | 50.3 | 55.6 |
| IG_3 | −3.4 | 61.2 | 52.3 | 55.5 |

TABLE 21
T-Student analysis indicates if there are significant differences in
the telomere shortening ratio by observing each treatment and time.
T-Student Analysis - Telomere shortening ratio - Standard conditions

| | p Value | Significance |
|---|---|---|
| Week 2 | | |
| Control vs. IG_1 | 8.77E−01 | NO |
| Control vs. IG_2 | 4.68E−02 | YES (*) |
| Control vs. IG_3 | 1.85E−01 | NO |
| Week 4 | | |
| Control vs. IG_1 | 3.30E−03 | YES (***) |
| Control vs. IG_2 | 1.11E−02 | YES (*) |
| Control vs. IG_3 | 2.00E−04 | YES (***) |
| Week 6 | | |
| Control vs. IG_1 | 1.37E−01 | NO |
| Control vs. IG_2 | 8.03E−01 | NO |
| Control vs. IG_3 | 9.97E−01 | NO |
| Week 8 | | |
| Control vs. IG_1 | 2.70E−01 | NO |
| Control vs. IG_2 | 1.40E−02 | YES (*) |
| Control vs. IG_3 | 4.96E−02 | YES (*) |

Significant differences are indicated in the “Significance” column.
From lowest to highest significance:
No: no significance;
Si (*): p < 0.05;
Si (**): p < 0.01;
Si (***): p < 0.001;
Yes (****): p < 0.0001

TABLE 22
Telomere shortening ratio (Median telomere length
(initial − final)/Population Doubling) for defined
treatments and times under oxidative stress conditions.
Telomere shortening ratio - Oxidative stress

| Treatment | Week 2 | Week 4 | Week 6 | Week 8 |
|---|---|---|---|---|
| H₂O₂ | 52.3 | 63.1 | 46.2 | 56.7 |
| IG_1 + H₂O₂ | 83.9 | 43.9 | 43.9 | 68.6 |
| IG_2 + H₂O₂ | 130.0 | 45.9 | 52.0 | 73.9 |
| IG_3 + H₂O₂ | 79.9 | 41.7 | 45.5 | 66.4 |

TABLE 23
T-Student analysis indicates if there are significant differences in
the telomere shortening ratio by observing each treatment and time.
T-Student Analysis -Telomere Shortening Rate - Oxidative condition

| | p Value | Significance |
|---|---|---|
| Week 2 | | |
| H₂O₂ vs. IG_1 + H₂O₂ | 1.24E−01 | NO |
| H₂O₂ vs. IG_2 + H₂O₂ | 5.57E−02 | NO |
| H₂O₂ vs. IG_3 + H₂O₂ | 3.49E−01 | NO |
| Week 4 | | |
| H₂O₂ vs. IG_1 + H₂O₂ | 3.65E−01 | NO |
| H₂O₂ vs. IG_2 + H₂O₂ | 4.03E−01 | NO |
| H₂O₂ vs. IG_3 + H₂O₂ | 3.20E−01 | NO |
| Week 6 | | |
| H₂O₂ vs. IG_1 + H₂O₂ | 3.32E−02 | YES (*) |
| H₂O₂ vs. IG_2 + H₂O₂ | 1.99E−01 | NO |
| H₂O₂ vs. IG_3 + H₂O₂ | 7.54E−01 | NO |
| Week 8 | | |
| H₂O₂ vs. IG_1 + H₂O₂ | 1.72E−01 | NO |
| H₂O₂ vs. IG_2 + H₂O₂ | 1.44E−01 | NO |
| H₂O₂ vs. IG_3 + H₂O₂ | 2.44E−01 | NO |

Significant differences are indicated in the “Significance” column. From lowest to highest significance: No: no significance; Si (*): p < 0.05; Si (**): p < 0.01; Si (***): p < 0.001; Si (****): p < 0.0001.

### 3.2.5 Observations and Conclusions

After determining the different variables of telomere length (median, 20th percentile and %<3 kbp) and the normalization of the data through population doubling, the following observations were made:

### Standard Conditions

After 2 weeks of treatment, IG_2 and IG_3 show a reduction in the telomere shortening ratio when compared to the control group. Although this effect is not apparent at week 4, the positive effect can be observed again at week 6 (for IG_2) and week 8 (for IG_2 and IG_3) suggesting a protective effect on telomeres. The reduction in shortening at week 8 was statistically significant.

### Oxidative Stress Conditions

After 4 weeks of treatment, the IG_1, IG_2 and IG_3 groups showed an improvement in reducing the telomere shortening rate. For week 6, the IG_1 and IG_3 groups continue to maintain this effect, where the IG_1 group also presents statistically significant differences.

### Example 4:—In Vivo Study on the Anti-Aging Effect on Caenorhabditis elegans of the Composition Analyzed in Example 2

A study was conducted on the anti-aging effect of a sample of the composition in accordance with the invention corresponding to the composition analyzed in example 2, in order to characterize the effect of said sample on life expectancy, mobility and antioxidant capacity in the in vivo model of worms Caenorhabditis elegans.

### 4.1. Materials and Methods

### Sample

A sample of the composition was evaluated in accordance with the invention corresponding to the composition analyzed in Example 2 (hereinafter also referred to as “Sample”).

In a first step, a stock solution was prepared with distilled water, performing serial dilutions to obtain, finally, different final concentrations in the plate of NGM medium (Nematode Growth Medium), used for the cultivation of C. elegans. The product was added on the surface of the agar.

### 4.2. Life Expectancy Trials

The trials were conducted with the wild strain of C. elegans (N2). Populations synchronized in age were cultured, collecting the embryos in the different culture dishes:
NGM culture medium (control)·
Culture medium NGM+Sample (0.05, 0.1, 0.5, 1, 5, 10, 25, 50, 100, 200 and 400 μg/mL; 1, 10 and 20 mg/mL).

The plates were incubated at 20° C. and nematode survival counts were performed in each condition, with periodic transfers from the study population every two days to a new medium. During this period, a viability count was carried out, to finally obtain survival curves in each condition.

The trials were conducted in duplicate.

### 4.3. Study of Mobility

The trials were conducted with the wild strain of C. elegans (N2). Age-synchronized worms were obtained from gravid adults, collecting embryos in plates with the different conditions:
NGM medium (control)
NGM Medium+Sample (0.5; 1, 5, 10, 100 μg/mL; and 0.5 and 1 mg/mL).

The worms were incubated at 20° C. until they reached a young adult age. At this point, worm samples (25/condition and assay) were taken to measure locomotion, which was determined by quantifying the number of sinusoidal curvatures over 40 seconds. To measure the dispersion of the worms, a total of 15 worms/condition were analyzed, depositing at the same time 5 worms in the center of the NGM plate and determining their position after 2 minutes (zone 1 closest to the center, 2 or 3 farther from the center).

### 4.4. Antioxidant Activity in C. elegans

The trials were conducted with the wild strain of C. elegans (N2). Age-synchronized worms were obtained from the incubation of gravid adults in the corresponding culture plates. The embryos were incubated at 20° C. under different culture conditions, NGM (control) and NGM supplemented with the sample at different doses:
NGM medium (control)
NGM+Vitamin C medium (10 μg/mL) (positive control)
NGM+Sample medium (0.05; 0.1; 0.5; 1, 5, 10, 25, 50, 100, 200 and 400 μg/mL).

The worms were incubated in the NGB medium (control) in the NGM+Vitamin C medium, as well as under the different conditions in the NGB+Sample medium at 20° C. Subsequently, 5-day-old adults were transferred to culture medium plates with hydrogen peroxide and incubated at 20° C. Finally, the survival of the worms in each condition was determined.

### 4.5. Statistical Analysis

The comparison of the survival and mobility results between the NGM control condition and the different treatments was carried out using the One-way Anova statistical test with a Tukey's post-test (Tukey's Multiple Comparison Test).

In the case of the analysis of survival curves, those conditions that provided positive results through the Log-rank test were statistically analyzed.

All these analyses were carried out with the statistical software GraphPad Prism version 7.0 of the American company GraphPad Software

### 4.6. Results

### Study of Life Expectancy

In order to evaluate the efficacy of the Sample on the longevity of C. elegans, life expectancy trials were carried out.

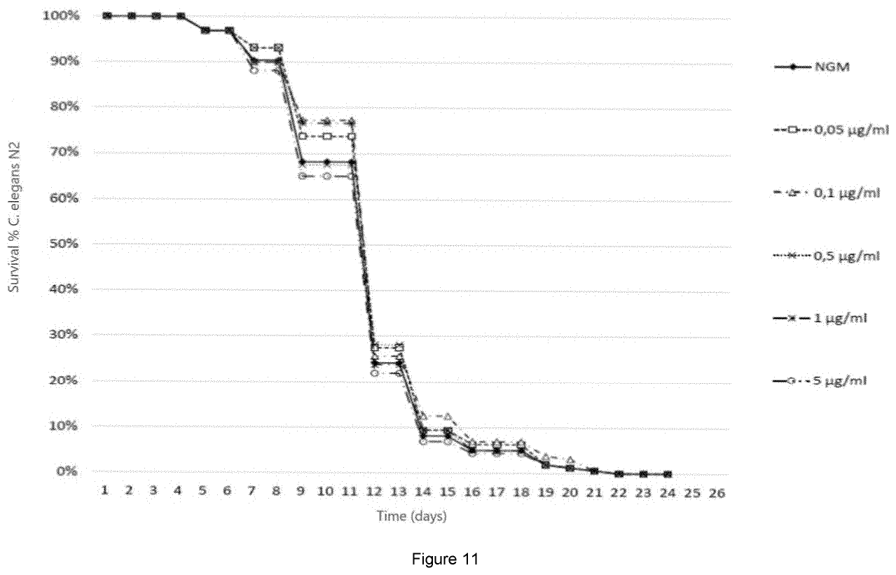

First, a series of doses were analyzed: 0.05; 1; 0.5; 1 and 5 μg/mL. FIG. 11 shows the result obtained with this dose range. It is noted that no dose was able to increase survival compared to the NGM control condition.

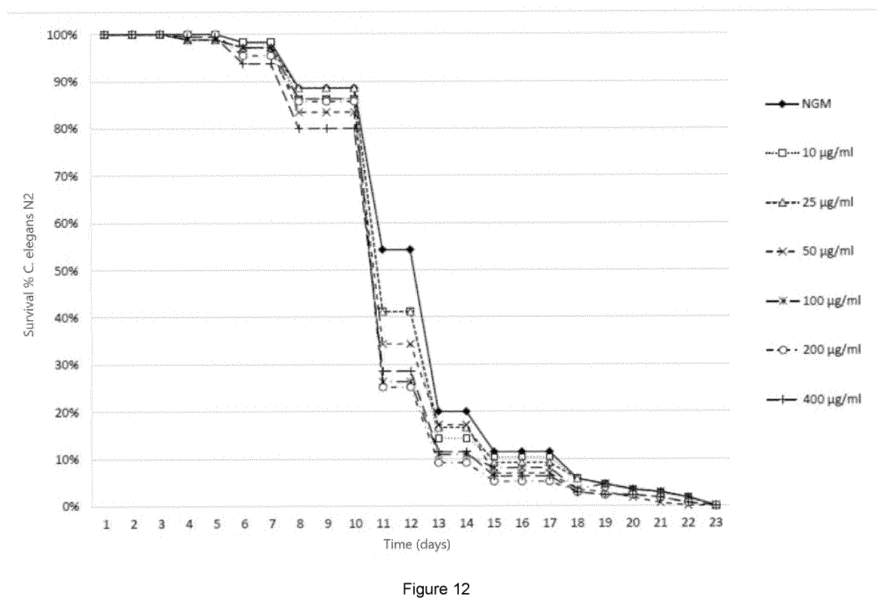

Secondly, a new life expectancy test was carried out with higher doses of the Sample (10, 25, 50, 100, 200 and 400 μg/mL). Again, it was also observed that none of the doses tested had any effect on the longevity of the nematode (FIG. 12).

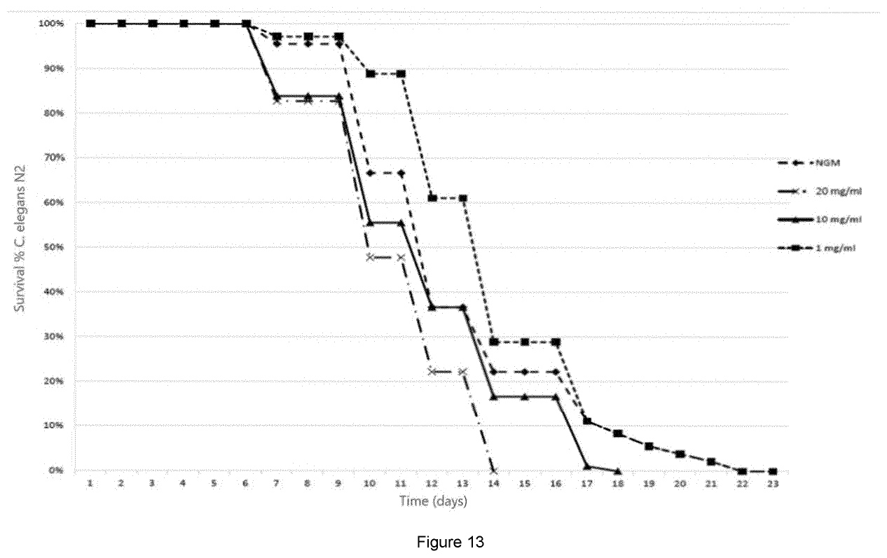

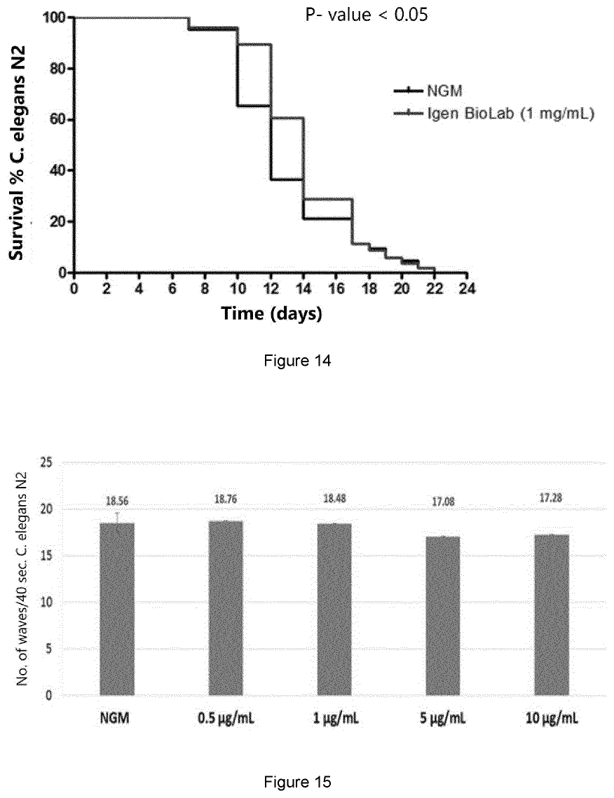

Finally, higher doses of the Sample, 1, 10 and 20 mg/mL, were evaluated in duplicate. The result indicated a significant effect of this product on the longevity of C. elegans at the dose of 1 mg/mL (P-value<0.05) (FIGS. 13 and 14, Table 24). The activity of the tested Sample produces an increase in average life expectancy of 2 days (increase of 16.6%) (Table 2 4). In the case of the higher doses, 10 and 20 mg/mL, some negative effect on the longevity of the worm was observed, since as the concentration of the product increases, the survival of the worm decreases.

FIG. 13 shows the effect of the high-dose Sample on the longevity of C. elegans N2. The value of average life expectancy (Time in which 50% of the population is alive) for the control condition (NGM) and the Sample at 1 mg/ml are indicated with dashed lines. FIG. 14 shows the survival curves of C. elegans (N2) obtained in populations treated with the Sample (1 mg/mL) using the GraphPad Prism software. On the other hand, Table 24 shows the statistical data of the survival curves of C. elegans after treatment with the Sample at the dose of 1 mg/ml

TABLE 24
Statistical data on the survival curves of C. elegans
after treatment with the Sample at a dose of 1 mg/mL

| | Average life | | P- value | |
|---|---|---|---|---|
| | expectancy | Final life | (treat. vs. | |
| Condition | (days) | expectancy | NGM) | Significant |
| NGM | 12 | 22 | | |
| Sample | 14 | 22 | 0.0264 | Yes (*) |
| (1 mg/mL) | | | | |

### Study of Mobility and Dispersion

The study of the locomotion of C. elegans after treatment with the Sample at different doses was carried out. Populations of adult worms were taken from different culture conditions, and the average number of sine waves was quantified for 40 seconds.

Initially, doses of 0.5 were tested; 1, 5 and 10 μg/mL. FIG. 15 represents the average sine waves for each of the different treatment doses. The treatment did not improve the mobility of C. elegans, as can be seen in FIG. 15, showing the average mobility of C. elegans populations with the Sample at different doses. The data are for a single trial.

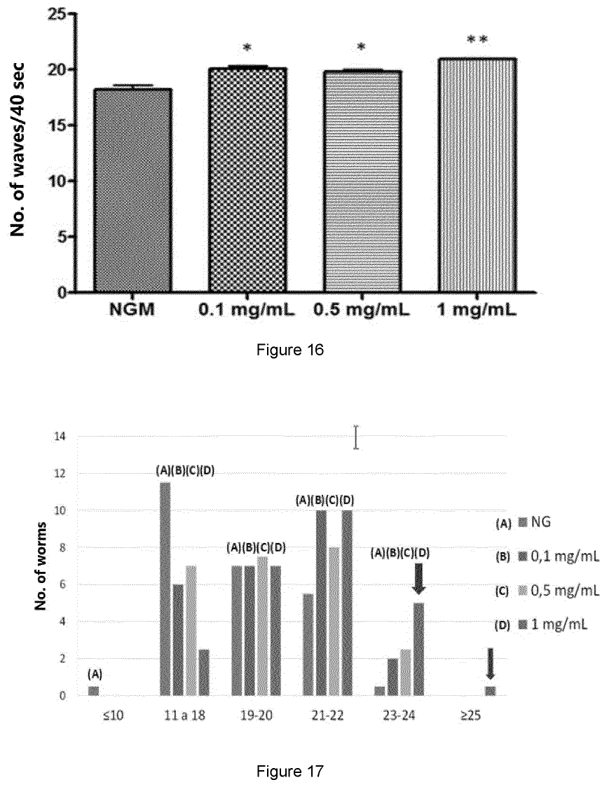

Subsequently, a higher dose of the Sample (0.1, 0.5 and 1 mg/mL) was tested in two independent trials. As can be seen in FIG. 16, showing the average mobility of C. elegans populations treated with the Sample, in this case, the product significantly increased the average movement of C. elegans at the three doses evaluated (0.1; 0.5 and 1 mg/mL), with the dose of 1 mg/mL showing the greatest increase (P≤0.01) with respect to control (NGM) (15.3% increase in mobility vs. control). The doses of 0.1 and 0.5 mg/mL caused an increase in mobility compared to the control condition of 10.3 and 8.9% respectively (P-value≤0.05).

In a next step, the frequency of curvatures was analyzed for each of the three previous doses (0.1, 0.5 and 1 mg/mL). To do this, groupings were made based on the number of worms that presented different number of sine waves/40 sec. FIG. 17 shows the distribution of nematode movement for each of the conditions analyzed. As can be seen, only worms with ≥25 waves were observed in the case of the 1 mg/ml dose; the rest of the conditions (0.1 and 0.5 mg/mL) showed worms with a number of curvatures between 23-24, this proportion being higher as the concentration of the product in the plate increases. The effective dose of 1 mg/mL had the highest number of worms with a number of curvatures between 23-24. This result reinforces the previous observation in which a positive effect of the product on the mobility of C. elegans is demonstrated.

Next, we analyzed whether treatment with the product increased the degree of dispersion of the worms with respect to control conditions. To do this, as described in materials and methods, the worms were placed in the center of the plate and their location (zone 1, 2 or 3) was measured after 2 minutes.

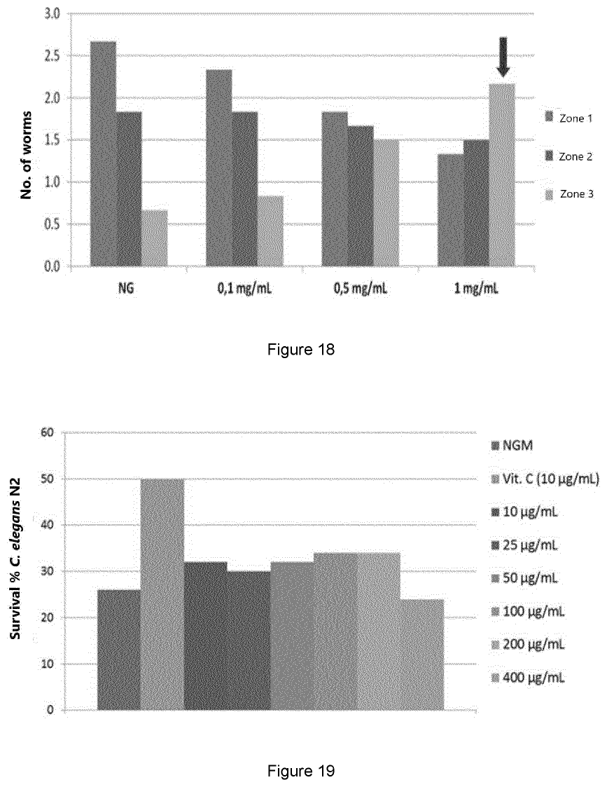

FIG. 18 showing the degree of dispersion of worms treated with the Sample represents the data obtained with adult worms treated with the product doses of 0.1; 0.5 and 1 mg/mL. It was determined that the dose of 1 mg/mL caused a greater accumulation of worms in zone 3 (most distal of origin), indicating a greater degree of dispersion of worms in this condition. In addition, it was observed that as we increase the concentration of the product, a greater number of worms accumulate in zone 3.

Therefore, these results indicate that treatment with the Sample is able to increase mobility and dispersion capacity in C. elegans.

### 4.7. Study of Antioxidant Activity in C. elegans

The study was conducted to determine whether treatment with the Sample improves sensitivity to acute oxidative stress in the nematode.

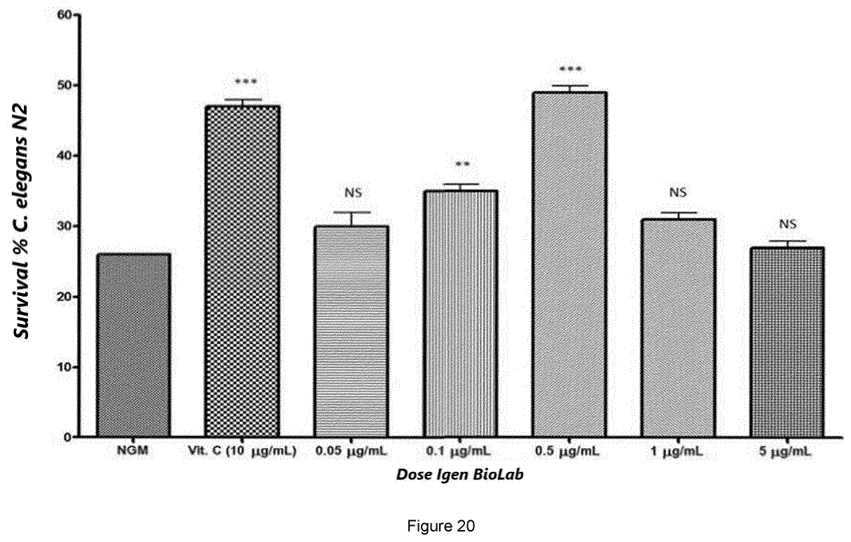

Initially, doses of 10, 25, 50, 100, 200 and 400 μg/mL of this product were evaluated. The results obtained with these doses are shown in FIG. 19, which represents for each condition the percentage of live worms accounted for after acute oxidative stress. FIG. 20 shows the antioxidant activity of the sample evaluated at different doses (10-400 μg/mL) in C. elegans N2. The percentages of survival after applying oxidative stress with H₂O₂. are shown. Vitamin C (10 μg/mL) was included as a positive control. The data are for a single trial.

Some protection exerted by the Sample was observed at these doses (slight increase in survival versus NGM control condition). However, the result was similar between the different doses analyzed, except for the dose of 400 μg/mL that showed a survival of the worms lower than the control. A possible reason for these results would be not being in the optimal dose range, so it was decided to evaluate lower doses of the Sample: 0.05; 0.1; 0.5, 1 and 5 μg/mL.

After subjecting the worms, previously treated with lower doses the Sample, to acute oxidative stress, a significant improvement in the survival of C. elegans was observed in those populations treated with the concentration of 0.1 and 0.5 μg/mL of the Sample. FIG. 20 shows the antioxidant activity of the Sample evaluated at different doses in C. elegans N2. The percentages of survival after applying oxidative stress with H₂O₂ are shown. Vitamin C (10 μg/mL) was included as a positive control. Data are for the mean of 2 independent trials (*** Significant at P≤0.001; ** Significant at P≤0.01. NS: Not significant.

Table 25 shows the percentage increase in survival and P-value obtained (ANOVA test) of each of the conditions of the Sample with respect to the NG control condition.

TABLE 25
percentage increase in survival and P-value obtained
(ANOVA test) of each of the conditions of the Sample
with respect to the NG control condition.

| | % increase survival | |
|---|---|---|
| Condition | vs control (NGM) | P-value |
| Vitamin C (10 μg/mL) | 21 | ≤0.001 |

| | | | |
|---|---|---|---|
| (0.05 | μg/mLg/mL) | 4 | NS |
| (0.1 | μg/mLg/mL) | 9 | ≤0.01 |
| (0.5 | μg/mLg/mL) | 23 | ≤0.001 |
| (1 | μg/mLg/mL) | 5 | NS |
| (5 | μg/mLg/mL) | 1 | NS |

The most effective dose was 0.5 μg/mL (23% increase in survival vs. control condition; P-value≤0.001) (Table 23). The dose of 0.1 μg/mL caused a 9% increase in survival vs control (P≤0.01) (Table 23).

These results indicate that the product is able to improve resistance to acute oxidative stress in vivo, indicating a potent antioxidant effect given the low protective dose.

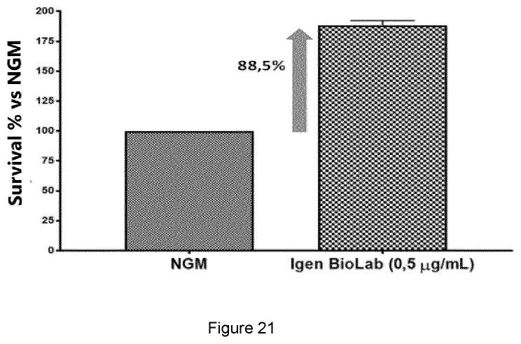

FIG. 21 represents the relative percentage increase in survival of C. elegans with the effective dose (0.5 μg/mL) of the Sample versus control condition, indicating an increase of 88.5%.

### 4.8. Summary of Test Results

The example shows a summary of the functional activity in C. elegans of the analyzed Sample. It can be seen that the Sample has been functionally characterized in the preclinical model of C. elegans, showing some effect on longevity (at high doses), a very significant effect on mobility and a high antioxidant activity (at very low doses).

### Example 5. Testing of a New Composition of the Invention

A new composition was prepared following the method described in Example 1, the components of the novel composition are described in Table 26

TABLE 26
Composition of sample 2 of the composition object of the invention.
The percentage of weight of each of the bacterial species comprising
the dry bacterial lysates described in this document is shown.

| Species | % weight |
|---|---|
| Bacillus clausii | 0.3 |
| Bacillus coagulans | 0.3 |
| Bacillus licheniformis | 5 |
| Bacillus mesentericus | 5 |
| Bacillus subtilis | 5 |
| Bifidobacterium bifidum | 2.5 |
| Bifidobacterium breve | 2.5 |
| Bifidobacterium lactis | 3 |
| Lactobacillus acidophilus | 3.7 |
| Lactobacillus bulgaricus | 3.6 |
| Lactobacillus casei | 5 |
| Lactobacillus fermentum | 3 |
| Lactobacillus reuteri | 3 |
| Lactobacillus rhamnosus | 3 |
| Saccharomyces cereviseae | 52 |
| Streptococcus thermophilus | 3 |

The oxidative stress activity and antiaging activity were evaluated in the same conditions as with Sample 1 in Example 3 and 4 and the results obtained were very similar.

These results strengthen the antiaging and anti-oxidative stress effect in aging of the composition of oral administration of the invention.

1. A composition of oral administration to prevent and delay aging, comprising lysates of probiotic microorganisms in a form of dry powder, in a following amount as a percentage by weight of a total:
16% of bacterial lysates of a genus Bacillus;
8% of bacterial lysates of a genus Bifidobacterium;
21% of bacterial lysates of a genus Lactobacillus;
52% of bacterial lysates of a genus Saccharomyces;
3% of bacterial lysates of a genus Streptococcus;
for use in the prevention and/or treatment of oxidative stress related with aging.

2. The composition for use, according to claim 1, wherein the bacterial lysates of the genus Bacillus are of a species that are selected from the group consisting of Bacillus clausii, Bacillus coagulans, Bacillus licheniformis, Bacillus pumilus, Bacillus mesentericus, Bacillus subtilis, Bacillus paralicheniformis, and combinations thereof.

3. The composition for use, according to claim 1, wherein the bacterial lysates of the genus Bifidobacterium are of a species that are selected from the group consisting of Bifidobacterium animalis subsp lactis, Bifidobacterium bifidum, Bifidobacterium breve, Bifidobacterium lactis, Bifidobacterium longum, Bifidobacterium infantis, Bifidobacterium animalis, and combinations thereof.

4. The composition for use, according to claim 1, wherein the bacterial lysates of the genus Lactobacillus are of a species that are selected from the group consisting of Lactobacillus acidophilus, Lactobacillus bulgaricus, Lactobacillus casei, Lactobacillus fermentum, Lactobacillus paracasei, Lactobacillus plantarum, Lactobacillus reuteri, Lactobacillus rhamnosus, Lactobacillus salivarius, Lactobacillus helveticus, Lactobacillus gasseri, Lactobacillus johnsonii, Lactobacillus brevis, Lactobacillus kefiri, and combinations thereof.

5. The composition for use, according to claim 1, wherein the bacterial lysates of the genus Saccharomyces are selected from the group consisting of Saccharomyces cerevisiae, Saccharomyces boulardii, and combinations thereof.

6. The composition for use, according to claim 1, wherein the bacterial lysates of the genus Streptococcus are of a species Streptococcus thermophilus, Streptococcus salivarius, and combinations thereof.

7. The composition for use, according to claim 1, comprising at least one additional component that is selected from the group consisting of Riboflavin, Vitamin C, Manganese, Selenium, Zinc, Vitamin D and Magnesium.

8. The composition for use, according to claim 1, wherein comprises a relative abundance, as a percentage by weight, comprised between:
1.50 to 9% of proteins from bacterial lysates of the genus Bacillus;
0.01% to 1.20% of proteins from bacterial lysates of the genus Bifidobacterium;
2% to 8% of proteins from bacterial lysates of the genus Lactobacillus;
49% to 99.00% of proteins from bacterial lysates of the genus Saccharomyces;
0.15% to 0.90% of proteins from bacterial lysates of the genus Streptococcus.

9. The composition for use, according to claim 1, as a food supplement.

10. The composition for use, according to claim 1, wherein the composition is administered an amount between 0.1 μg to 200 mg of the composition and 2 to 6 times a day orally.

11. The composition for use, according to claim 1, wherein the composition is presented in the form of dry powder inside watertight sachets or encapsulated in gelatin capsules.

12. A pharmaceutical composition comprising an effective pharmaceutical amount of the composition, according to claim 1, and a pharmaceutically acceptable excipient for use in the prevention and/or treatment of oxidative stress related with aging.
